(19) United States
(12) Patent Application Publication
(10) Pub. No.: US 2021/0236507 A1
(43) Pub. Date: Aug. 5, 2021

(54) METHODS AND PHARMACEUTICAL COMPOSITIONS FOR PREVENTING RELAPSE IN A PERSON WITH AN ADDICTION USING BETA ADRENERGIC RECEPTOR ANTAGONIST AND MUSCARINIC RECEPTOR ANTAGONIST COMBINATIONS

(71) Applicant: MAGNOLIA CNS, LLC, Ridgeland, MS (US)

(72) Inventor: Thomas P. DOOLEY, Pinson, AL (US)

(21) Appl. No.: 17/083,783

(22) Filed: Oct. 29, 2020

Related U.S. Application Data
(60) Provisional application No. 62/970,245, filed on Feb. 5, 2020.

(51) Int. Cl. A61K 31/5386; A61K 31/165; A61K 31/404; A61K 31/138; A61K 31/353; A61P 25/36

(52) U.S. Cl. CPC A61K 31/5386; A61K 31/165; A61K 31/404; A61K 45/06; A61K 31/353; A61P 25/36; A61K 31/138

(57) ABSTRACT

The present invention is directed to pharmaceutical compositions containing at least one beta adrenergic receptor antagonist and at least one antiemetic muscarinic receptor antagonist and methods for preventing relapse and/or reducing the risk of relapse in subjects having alcohol and/or drug addiction and who are abstinent to alcohol and/or drug use. The present invention encompasses pro re nata (“as needed”) treatment of the symptoms of anxiety incident to substance use disorder treatment and/or recovery, thereby reducing the risk of relapse to alcohol and/or drug use in a subject, in an embodiment, the invention encompasses combinations of standard-of-care medicines for addiction and such pharmaceutical compositions for enhanced effectiveness of the treatment for alcohol and/or drug addiction.

### STATEMENT REGARDING PRIOR DISCLOSURES BY THE INVENTOR OR A JOINT INVENTOR QUALIFYING FOR THE GRACE PERIOD PROVIDED UNDER 35 USC § 102(b)(1)

The applicant submits that the following disclosures qualify under the grace period provided as noted above:

Ashley B. Benjamin and Thomas P. Dooley, “Anxiolytic benefits of compounded Atenolol-Scopolamine in eight patients in psychiatry” Personalized Medicine in Psychiatry, doi.org/10.1016/j.pmip.2019.10.001, published 1 Nov. 2019.

Thomas P. Dooley, Ashley B. Benjamin, and Ty Thomas, “Treating anxiety with a beta blocker-antimuscarinic combination A review of compounded Atenolol—Scopolamine”, Clinical Psychiatry 5(3):63, doi.org/10.36648/2471-9854.5.1.63, published 20 Dec. 2019.

### BACKGROUND OF THE INVENTION

### Field of the Invention

The invention encompasses compositions and methods for preventing and/or reducing the risk of relapse (or recidivism) to alcohol and/or drug use and/or addiction comprising administering to a subject being treated for a substance use disorder (SUD), alcohol use disorder (AUD), opioid use disorder (OUD), or who is a recovering addict, a pharmaceutical composition consisting essentially of a combination of at least one beta adrenergic receptor antagonist agent and/or pharmaceutically acceptable salts thereof, and at least one antiemetic muscarinic receptor antagonist agent and/or pharmaceutically acceptable salts thereof, in a therapeutically effective amount to reduce symptoms of anxiety in the subject, whereby the administration reduces the risk of relapse and/or prevents relapse to substance abuse and/or addiction (i.e., alcohol and/or drug use).

### Description of Related Art

The abuse of alcohol, prescription drugs, and illegal drugs (e.g, opioids/opiates and cocaine) are major mental health care concerns. The repetitive abuse of these chemicals can produce physiologic dependence, tolerance, and addiction. The symptoms of sudden withdrawal depend upon the abused substance, the impairment of neurological and neuroendocrine pathways, as well as visceral and peripheral somatic organ impairment. The withdrawal from addictive substances produces an array of acute symptoms, many of which overlap with the symptoms of panic and acute anxiety. Delirium tremens (DTs) occurs in some alcoholics upon abrupt cessation of drinking alcohol. The symptoms of alcohol-related DTs are very similar to those of panic attacks, and are in part related to beta adrenergic effects. The DTs can have serious and even life-threatening consequences. The standards-of-care for DTs are oral benodiazepines. Withdrawal from opioid and/or opiate addiction is physiologically distinct from alcohol withdrawal, albeit some characteristics are common to both.

Substance use disorder (SUD), alcohol use disorder (AUD), opioid use disorder (OUD) are defined in the Diagnostic and Statistical Manual of Mental Disorders, Fifth Edition, or DSM-5. For instance, opioid use disorder (OUD), is a chronic lifelong disorder, with serious potential consequences including disability, relapses, and death. OUD is defined in the DSM-5 assessment criteria as a problematic pattern of opioid use leading to clinically significant impairment or distress. OUD has also been referred to as “opioid addiction.” Opioid use disorder can be medically treated with pharmacotherapies and/or non-medical interventions, and often within the context of residential treatment centers.

Maintenance-based treatments of addiction are associated with the development of a pharmacological steady-state such that addictive substances are no longer sought for reward (e.g, euphoria or altered mood or cognition) and/or relief (e.g, adverse symptoms of withdrawal). Maintenance treatments of addiction are also designed to mitigate against the risk of overdose, Depending on the circumstances of a given case, a care plan including maintenance treatments can be time-limited or can remain in place lifelong. Integration of pharmacotherapy via maintenance treatments with psychosocial treatments (e.g, psychotherapy, counseling, spiritual development, and/or “step programs”) generally is associated with the best clinical results. Maintenance treatments can be part of an individual's treatment plan in abstinence-oriented recovery activities.

Treatments include, but are not limited to, counseling and often coupled with the administration of drugs that act as antagonists at the opioid receptors, opioid receptor agonist, or mixed opioid agonist/antagonists, and/or drugs that try to reduce the craving associated with drug addiction, Current standard-of-care treatments involve replacing the addictive drug with a substitution of an opioid receptor agonist or mixed agonist/antagonist medicine, A therapeutic approach consists of the use of an opioid receptor antagonist to block the effect of the agonist.

Medications for OUD, such as buprenorphine, buprenorphine combined with naloxone, naltrexone, and methadone are FDA approved first-line, standard-of-care medicines in maintenance-based treatments of opioid addiction.

Most treatments for addiction start with detoxification and medically managed withdrawal, often considered the first stage of treatment, Detoxification, the process by which the body clears itself of drugs, is designed to manage the acute and potentially dangerous physiological effects of stopping drug use.

Because it is often accompanied by unpleasant and potentially fatal side effects stemming from withdrawal, detoxification is often managed with medications administered by a physician in an inpatient or outpatient setting; therefore, it is referred to as “medically managed withdrawal” Medications are available to assist in the withdrawal from, for example, opioids, benzodiazepines, alcohol, nicotine, barbiturates, and other sedatives.

Relapse following detoxification alone is extremely common, and therefore detoxification rarely constitutes an adequate treatment of substance dependence on its own. However, it is a first step for many forms of longer-term abstinence-oriented treatment. Both detoxification with subsequent abstinence-oriented treatment and substitution maintenance treatment are essential components of an effective treatment system for people with opioid dependence/addiction.

Drug or alcohol treatment failures (i.e., relapse) can result in overdose deaths or other causes of death as a consequence of drug or alcohol use (e.g, traumatic accidents or suicide).

Abstinence-oriented treatment is the most common form of addiction treatment. This approach, which is based on the disease model of addiction, assumes that people with substance use disorders are always at risk of a relapse. The fear of completely ceasing use of alcohol and/or drugs can be intimidating, overwhelming and, thus, a source of anxiety or stress that results in anxiety.

Present therapies used to treat opioid addiction have major limitations leading to a very high rate of relapse to opioid use. The contributing neurochemical events in opioid abuse and addiction are complex. As a result, single acting neuropharmacological approaches do not appear to be sufficient to overcome addiction, What is more, the drugs currently used in opioid addiction treatments can have significant side-effects themselves, limiting their utility.

The most common or “standard-of-care” medications used in the abstinence-oriented treatment and/or maintenance treatments (substitution) for opioid addiction include, for example, methadone; buprenorphine; a combination of buprenorphine and naloxone; and naltrexone. Each medication works in a different way and has its own risks and benefits. Once started, the medication can be safely taken for years and help patients manage addiction so that they can recover.

Fundamental tenants of abstinence-oriented recovery programs hold that alcoholism and drug addiction is not curable, but the diseases may be arrested. The long-lasting, chronic nature of many addictions and high rates of relapse present a considerable challenge for effective treatment of drug and alcohol addiction to achieve long-term abstinence.

Multiple factors contribute to the likelihood of relapse from abstinence in patients with a substance use disorder (SUD).). The one-year relapse outcomes (i.e., “treatment failure”) following treatment for alcohol, opioids, cocaine, marijuana, and nicotine can be as high as 85% [1, 2]. Less than 35% of alcohol- and opiate-dependent patients who were abstinent when discharged from treatment for substance abuse remained abstinent at one year [2]. Thus, the prevention of relapse is of great concern, and for which the medical and non-medical standards-of-care still provide minimal-to-moderate effectiveness.

Anxiety and stress that produces anxiety are key factors, among other factors (e.g., drug-related cues and depression), that are associated with the likelihood of relapse to substance abuse [2, 3]. These two factors (anxiety and stress) are predictive of relapse and vulnerability to addiction, and thus one can also reasonably assert that these factors are causative in relapse in some patients.

The association between anxiety and/or stress and relapse to SUDs has been established for many types of substances:

Alcohol [5-9]: Trait anxiety, assessed using the State-Trait Anxiety Inventory, was a significant predictor for relapse in women and men, and higher trait anxiety indicated a higher probability of relapse [5], In the clinical setting with AUD patients, stress and alcohol-cue exposures increase subjective anxiety and alcohol craving [6].

With regard to comorbidity, the odds ratio between AUD and any anxiety disorder is approximately 2.1 to 3.3 [9] Smith and Randall noted, “The reported rates of self-medication in clinical samples of people with both types of disorders have ranged from 50 to 97 percent, with the highest rates among people with phobias . . . ”[9]. In alcohol-dependent men, 35.8% have a comorbid anxiety disorder, whereas the rate is even higher in alcohol-dependent women, where 60.7% have a comorbid anxiety disorder [9]. Oliva and coworkers stated, “ . . . females with AUD suffered from higher levels of state/trait anxiety and depression than males.” [7]. Furthermore, “ . . . both state and trait anxiety levels assessed 6 months after discharge predicted alcohol relapse in the following 6 months in males . . . ” [7].

In general, emotional and environmental factors (conditioning stimuli) were listed among the causes of relapse. For example, it is known that specific stress conditions such as loss of work and economic difficulties or stimuli predictive of the presence of alcohol previously associated with its use, may strongly facilitate relapse in detoxified former alcoholics.

Opioids [10, 11]: The opioid receptor antagonist naltrexone is one of the FDA-approved medications for the prevention of relapse in OUD by blocking the receptor and thus opioid euphoria. In a clinical setting, anxiety assessed as a “tense, anxious, and/or jittery” condition, was significantly elevated when naltrexone-treated patients were exposed to stress and drug-cue conditions [10]. Furthermore, stress and drug-cue conditions also increased fear, heart rate, blood pressure (systolic and diastolic), and drug-cravings, and decreased the relaxed state . . . a group of symptoms consistent with anxiety in general Thus, stress and drug cues may produce both sympathetic and parasympathetic symptoms of anxiety, thus eliciting opioid cravings that contribute to treatment failures in these antagonist-treated patients [10].

Marijuana [12]: Bonn-Miller and Moos evaluated the rate of relapse to marijuana use at 12-months post-treatment [12]. They concluded that “Anxiety symptoms at discharge predicted relapse to marijuana use at 12-month follow-up . . . ” [12].

Nicotine [13, 14]: Anxiety Sensitivity, the cognitive fear of future development of symptoms of acute anxiety episodes (e.g., palpitations), is associated with early relapse to tobacco/nicotine use [14], The Anxiety Sensitivity Index was associated with early relapse [14].

Some patients anticipate future episodes of panic or acute anxiety based upon his/her history of encounters with a known “trigger” circumstance for panic and/or anxiety. A trigger circumstance might be associated with substance abuse, for example, alcohol-associated cue exposure, drug-associated cue exposure, and the like.

External triggers are people, places, activities and objects that elicit thoughts or cravings associated with substance use. Individuals in recovery can stay away from the dangers of external triggers and avoid triggers that remind them of past drug use. They should also be prepared to fight thoughts, temptations and cravings when they are in triggering situations.

Internal triggers are more challenging to manage than external triggers. They involve feelings, thoughts or emotions formerly associated with substance abuse, Emotions that can act as internal triggers may include fear and anxiousness. When internal triggers arise, they can lead to questionable behaviors that deter recovery progress and may cause individuals to crave and be tempted to use alcohol and/or drugs.

Theoretical positions exist to explain the persistence of addictive behavior and vulnerability to relapse associated with drug and alcohol addiction, homoeostatic hypotheses relate relapse risk to neuroadaptive changes and disruption of neuroendocrine homeostasis that are thought to underlie anxiety, mood dysregulation and somatic symptoms that accompany acute withdrawal, and that can persist for considerable periods of time during what has been referred to as the “protracted withdrawal” phase.

This view, therefore, implicates alleviation of discomfort from the symptoms of anxiety which are manifested in a patient during treatment and recovery for alcohol and/or drug addiction which contribute to the likelihood of relapse to alcohol and/or drug use.

The pharmaceutical standards-of-care in routine psychiatric care of panic attack (PA), panic disorder (PD), anxiety, and anxiety-related disorders, and the symptoms thereof, involve two key aspects: (1) prophylaxis, rather than treatment of the symptoms per se; and (2) the medications are routinely given as chonic, oral “maintenance” medications for persistent use (i.e., chronic prophylaxis), rather than administration “as needed” (p.r.n.), for example, at the time of episodes of symptoms (i.e. acute therapy).

Pharmacological approaches to the treatment of anxiety and the symptoms of anxiety in patients who are undergoing treatment for a substance use disorder may involve the use of benzodiazepine drugs which may help manage the psychological aspects of withdrawal, detoxification and compliance with standard-of-care therapeutics, But, their effectiveness varies widely and may be contraindicated for use in patients having issues with alcohol and/or drug dependency, or for which benzodiazepines are not suitable.

What is more, benzodiazepines may be contraindicated for concomitant, coincident and/or ancillary use with some standard-of-care medicines for alcohol and/or drug addiction, alcohol and/or drug withdrawal, or for opioid addiction and/or withdrawal.

Benzodiazepines can produce disabling psychic and somatic side effects, such as sedation, lethargy, chemical dependence, tolerance, and sexual dysfunction. As a result of these negative side effects and potential for abuse, benzodiazepines are classified by the U. S. Food and Drug Administration (FDA) as Schedule 4 (IV) “Controlled Substances”. In 2016 the US FDA issued a warning against the coincident use of benzodiazepines and opioids. Benzodiazepines are contributors to the current “Opioid Crisis” that claimed the lives of ca. 72,000 persons in the USA in 2017. In 2020 the US FDA further increased the warning status for benzodiazepines, in particular concerning potential for abuse. Benzodiazepines (e.g. alprazolam, clonazepam) pose significant risks of side effects such as dependence (i.e., adverse withdrawal reactions), memory impairment, addiction, misuse, and other adverse events such as falls or other accidents.

There is a need for fast-acting, effective, non-dependent and non-addicting anxiolytic treatments, in lieu of benzodiazepines, for alleviation of the symptoms of anxiety and/or panic in subjects undergoing abstinence-oriented treatments for alcohol and/or drug addiction.

In spite of the abundance and availability of numerous oral psychiatric pharmaceuticals, there is a need for a pharmaceutical composition for the treatment of symptoms of anxiety and panic in subjects who are being treated for a substance use disorder (SUD), alcohol and/or drug addiction, including subjects who have been detoxified and are compliant with a standard-of-care medication, such as an opioid deterrent standard-of-care medication, and/or in subjects who are afflicted with opioid use disorder, specifically, as well as subjects who have been treated for drug addiction and/or are recovering addicts. There is a need for a pharmaceutical composition for the treatment of symptoms of anxiety and panic, thereby preventing relapse and/or reducing the risk of relapse in subjects having an SUD and who are abstinent to alcohol and/or drug use.

### BRIEF SUMMARY OF THE INVENTION

The present invention provides compositions and methods for preventing relapse and/or reducing the risk of relapse to substance use in a subject having a substance use disorder (SUD), The method comprises administering to a subject having an SUD, a pharmaceutical composition consisting essentially of at least one beta adrenergic receptor antagonist agent and/or pharmaceutically acceptable salts thereof, and at least one antiemetic muscarinic receptor antagonist agent and/or pharmaceutically acceptable salts thereof, in amounts therapeutically effective for symptomatic treatment of panic and/or anxiety incident to treatment and/or recovery from addiction in the subject/patient having an SUD, thereby preventing and/or reducing the risk of relapse to substance use in the subject.

The present invention provides compositions and methods for preventing relapse and/or reducing the risk of relapse to substance use, the methods comprising administering to the subject or patient having an SUD, a pharmaceutical composition consisting essentially of at least one beta adrenergic receptor antagonist agent and/or pharmaceutically acceptable salts thereof, and at least one antiemetic muscarinic receptor antagonist agent and/or pharmaceutically acceptable salts thereof, in amounts therapeutically effective for p.r.n. treatment of the symptoms of panic and/or anxiety incident to treatment and/or recovery from addiction in the subject, thereby preventing and/or reducing the risk of relapse to substance use in the subject.

The present invention provides compositions and methods for preventing relapse and/or reducing the risk of relapse to substance use in a subject having an alcohol and/or drug addiction. The methods comprise administering to a subject having an alcohol and/or drug addiction, a pharmaceutical composition consisting essentially of at least one beta adrenergic receptor antagonist agent and/or pharmaceutically acceptable salts thereof, and at least one antiemetic muscarinic receptor antagonist agent and/or pharmaceutically acceptable salts thereof, in amounts therapeutically effective for symptomatic treatment of panic and/or anxiety incident to treatment and/or recovery from addiction in the subject, thereby preventing and/or reducing the risk of relapse to alcohol and/or drug use in the subject.

Compositions and methods for treating and/or preventing symptoms of anxiety in subjects being treated for opiate addiction are also provided. The methods comprise administering to a subject having an opiate addiction, a pharmaceutical composition consisting essentially of at least one beta adrenergic receptor antagonist agent and/or pharmaceutically acceptable salts thereof, and at least one antiemetic muscarinic receptor antagonist agent and/or pharmaceutically acceptable salts thereof, in amounts therapeutically effective for symptomatic treatment of panic and/or anxiety in the subject/patient having an opiate addiction, thereby preventing and/or reducing the risk of relapse to opiate use in the subject.

The present invention provides compositions and methods for treating alcohol and/or drug addiction, comprising administering a combination of a standard-of-care medicine for treating alcohol and/or drug addiction and a pharmaceutical composition consisting essentially of at least one beta adrenergic receptor antagonist agent and/or pharmaceutically acceptable salts thereof, and at least one antiemetic muscarinic receptor antagonist agent and/or pharmaceutically acceptable salts thereof, in amounts therapeutically effective for the treatment of symptoms of panic and/or anxiety incident to treatment and/or recovery from addiction in the subject.

What we therefore believe to be comprised by our invention may be summarized inter aa in the following words.

The invention encompasses pharmaceutical compositions for pro re nata (p.r.n.) treatment and/or prevention of relapse in a subject having a substance use disorder, the pharmaceutical compositions consisting essentially of at least one beta adrenergic receptor antagonist agent and/or pharmaceutically acceptable salts thereof, and at least one antiemetic muscarinic receptor antagonist agent and/or pharmaceutically acceptable salts thereof, together with one or more pharmaceutically acceptable excipients, wherein the at least one beta adrenergic receptor antagonist agent and the at least one antiemetic muscarinic receptor antagonist agent are combined in amounts therapeutically effective for p.r.n, treatment of the symptoms of panic and/or anxiety in the subject and wherein the pharmaceutical composition is in a form for mucosal, sublingual, buccal, rectal, vaginal, nasal, or oral p.r.n. administration by the subject anticipating and/or experiencing symptoms of anxiety incident to SUD treatment and/or recovery, alcohol and/or drug addiction, whereby administering the pharmaceutical composition reduces the risk of relapse to alcohol and/or drug use in the subject.

In an aspect, the pharmaceutical composition is in a form selected from the group consisting of a spray, an elixir, a solution, a suspension, an emulsion, a gel, a cream, a gum, a powder, a tablet, a capsule, a troche, a suppository, a pill, and a film.

In an embodiment, the form is a liquid or semi-solid form comprising at least one penetration-enhancing solvent selected from the group consisting of ethanol, glycerol, propylene glycol, ethoxydiglycol, and dimethylsulfoxide or wherein the form is a solid form comprising at least one component selected from the group consisting of mannitol, a monosaccharide, a disaccharide, a sweetener, a citrate buffer or citric acid, a bicarbonate buffer, a phosphate buffer, a binding agent, a gelatin, a collagen, and a preservative.

In an aspect of the pharmaceutical composition of the invention, the at least one beta adrenergic receptor antagonist agent is selected from the group consisting of propranolol, atenolol, alprenolol, acebutolol betaxolol, bisoprolol, bucindolol, celiprolol, nadolol sotalol, esmolol, carteolol, carvedilol, mepindolol, nebivolol oxprenolol, penbutolol, pindolol, landiolol, metoprolol, timolol labetolol, and/or pharmaceutically acceptable salts thereof, and Eucommia extract, an active pharmaceutical ingredient of Eucommia, and/or pharmaceutically acceptable salt thereof, and the at least one antiemetic muscarinic receptor antagonist agent is selected from the group consisting of scopolamine, diphenhydramine, meclizine, buclizine, cyclizine, hydroxyzine, pirenzepine, benztropine (benzatropine), butylscopolamine, methylscopolamine, doxylamine, promethazine, trihexyphenidyl, orphenadrine, and/or pharmaceutically acceptable salts thereof.

In a further aspect of the pharmaceutical composition of the invention, the at least one beta adrenergic receptor antagonist agent and/or pharmaceutically acceptable salts thereof, is in an amount of about 10 to 100 mg/dose, and the at least one antiemetic muscarinic receptor antagonist agent and/or pharmaceutically acceptable salts thereof, is scopolamine in an amount of about 0.05 to 1.0 mg/dose, or wherein the at least one antiemetic muscarinic receptor antagonist agent is not scopolamine and is in an amount of about 10 to 100 mg/dose.

The pharmaceutical composition of the invention wherein at least one antiemetic muscarinic receptor antagonist agent is scopolamine, and wherein the at least one beta adrenergic receptor antagonist agent and the at least one antiemetic muscarinic receptor antagonist agent are combined in amounts therapeutically effective for p.r.n. symptomatic treatment, for example, in a ratio which falls within a range of 300:1 to 40:1 (beta adrenergic receptor antagonist agent:antiemetic muscarinic receptor antagonist agent).

The invention encompasses methods for reducing the occurrence of relapse to alcohol and/or drug use in a subject comprising, administering to a subject having an SUD and is abstinent to substance use, a pharmaceutical composition consisting essentially of a combination of at least one beta adrenergic receptor antagonist agent and/or pharmaceutically acceptable salts thereof, and at least one antiemetic muscarinic receptor antagonist agent and/or pharmaceutically acceptable salts thereof, together with one or more pharmaceutically acceptable excipients, wherein the at least one beta adrenergic receptor antagonist agent and the at least one antiemetic muscarinic receptor antagonist agent are combined in amounts therapeutically effective for pro re nata (p.r.n.) treatment of symptoms of anxiety, thereby reducing the risk of relapse to substance use in the subject.

The method of the invention encompasses the use of a pharmaceutical composition which consists essentially of at least one beta adrenergic receptor antagonist agent selected from the group consisting of propranolol, atenolol, pindolol nadolol, nebivolol, and/or pharmaceutically acceptable salts thereof, and Eucommia extract, and at least one antiemetic muscarinic receptor antagonist agent selected from the group consisting of scopolamine, diphenhydramine, promethazine, meclizine, hydroxyzine, and/or pharmaceutically acceptable salts thereof.

In an embodiment, the at least one beta adrenergic receptor antagonist agent is propranolol, atenolol, pindolol, nadolol nebivolol, and/or pharmaceutically acceptable salts thereof, in an amount of about 10 to 100 mg/dose for an adult, and the at least one antiemetic muscarinic receptor antagonist agent is scopolamine, and/or pharmaceutically acceptable salts thereof, in an amount of about 0.05 to 1 mg/dose for an adult.

In an aspect, administration occurs when the subject anticipates symptoms of anxiety, or at the time of a trigger circumstance for anxiety, a drug-associated cue, an alcohol-associated cue, or during symptoms of anxiety.

In an aspect, the beta adrenergic receptor antagonist agent is intended to address the sympathetic symptoms of anxiety (especially the cardiovascular symptoms), and the antiemetic muscarinic receptor antagonist agent is intended to address the parasympathetic symptoms of anxiety, wherein the combination of APIs reduces anxiety symptoms, and the anxiolytic effects thus reduce or abrogate the risk of SUD relapse in patients affected by alcohol or drug dependence and/or addiction.

In an aspect, the subject having a substance use disorder is being detoxified from alcohol and/or drugs, or has been detoxified from alcohol and/or drugs and is abstinent to alcohol and/or drug use.

In a further aspect, the subject having a substance use disorder is addicted to alcohol and/or one or more drugs selected from opioids, opiates, and cocaine.

In an aspect of the invention, the subject is coincidentally receiving a standard-of-care medicine for alcohol addiction and/or drug addiction, wherein the standard-of-care medicine is a deterrent to alcohol or drug use.

In a further aspect, the standard-of-care medicine is selected from the group consisting of an opioid antagonist, an opioid agonist, and a mixed opioid agonist/antagonist.

The invention encompasses methods of treating a subject with an opioid addiction comprising administering a combination of at least one standard-of-care medicine for opioid addiction and a pharmaceutical composition consisting essentially of a combination of at least one beta adrenergic receptor antagonist agent and/or pharmaceutically acceptable salts thereof, and at least one antiemetic muscarinic receptor antagonist agent and/or pharmaceutically acceptable salts thereof, together with one or more pharmaceutically acceptable excipients, wherein the at least one beta adrenergic receptor antagonist agent and the at least one antiemetic muscarinic receptor antagonist agent are combined in amounts therapeutically effective for p.r.n. symptomatic treatment of a subject and herein the pharmaceutical composition is in a form for mucosal, sublingual, buccal, rectal, vaginal, nasal, or oral p.r.n. administration by the subject anticipating and/or experiencing symptoms of anxiety associated with opioid addiction, opioid withdrawal, trigger circumstances or drug-associated cues.

In an aspect, the method encompasses the use of pharmaceutical compositions consisting essentially of at least one beta adrenergic receptor antagonist agent and/or pharmaceutically acceptable salts thereof, in an amount of about 10 to 100 mg/dose, and at least one antiemetic muscarinic receptor antagonist agent and/or pharmaceutically acceptable salts thereof, in an amount of about 0.05 to 100 mg/dose.

The invention relates to methods for treating alcohol and/or drug addiction comprising administering a combination of a standard-of-care medicine and a pharmaceutical composition consisting essentially of a combination of at least one beta adrenergic receptor antagonist agent and/or pharmaceutically acceptable salts thereof, and at least one antiemetic muscarinic receptor antagonist agent and/or pharmaceutically acceptable salts thereof, together with one or more pharmaceutically acceptable excipients, wherein the at least one beta adrenergic receptor antagonist agent and the at least one antiemetic muscarinic receptor antagonist agent are combined in amounts therapeutically effective for pro re nata (p.r.n.) symptomatic treatment of panic and/or anxiety in the subject and wherein the pharmaceutical composition is in a form for mucosal, sublingual, buccal, rectal, vaginal, nasal, or oral administration by the subject.

In an aspect, administration of the pharmaceutical composition occurs when the subject anticipates symptoms of anxiety, or at the time of a trigger circumstance for anxiety, a drug-associated cue, an alcohol-associated cue, or during symptoms of anxiety.

In an aspect, the method encompasses the use of pharmaceutical compositions consisting essentially of at least one beta adrenergic receptor antagonist agent selected from the group consisting of propranolol, atenolol, pindolol nadolol, nebivolol, and/or pharmaceutically acceptable salts thereof, and Eucommia extract, and at least one antiemetic muscarinic receptor antagonist agent selected from the group consisting of scopolamine, diphenhydramine, promethazine, meclizine, hydroxyzine, and/or pharmaceutically acceptable salts thereof.

In an aspect, the method encompasses the use of pharmaceutical compositions consisting essentially of at least one beta adrenergic receptor antagonist agent which is propranolol, atenolol, pindolol, nadolol, nebivolol, and/or pharmaceutically acceptable salts thereof, in an amount of about 10 to 100 mg/dose for an adult, and at least one antiemetic muscarinic receptor antagonist agent which is scopolamine, and/or pharmaceutically acceptable salts thereof, in an amount of about 0.05 to 1.0 mg/dose for an adult, and wherein the doses are lower for an adolescent or child.

The invention encompasses a combination for treating alcohol and/or drug addiction comprising a standard-of-care medicine for alcohol and/or drug addiction and a pharmaceutical composition consisting essentially of at least one beta adrenergic receptor antagonist agent and/or pharmaceutically acceptable salts thereof, and at least one antiemetic muscarinic receptor antagonist agent and/or pharmaceutically acceptable salts thereof, wherein the at least one beta adrenergic receptor antagonist agent and the at least one antiemetic muscarinic receptor antagonist agent are combined in amounts therapeutically effective for p.r.n. treatment of symptoms of anxiety in a subject and wherein the pharmaceutical composition is in a form for mucosal, sublingual, buccal, rectal, vaginal, nasal, or oral administration by the subject anticipating and/or experiencing symptoms of anxiety incident to SUD treatment and/or recovery, trigger circumstances and drug associated cues.

In an aspect of the combination, the pharmaceutical composition is in a liquid or semi-solid form comprising at least one penetration-enhancing solvent selected from the group consisting of ethanol, glycerol, propylene glycol, ethoxydiglycol, and dimethylsulfoxide, or a solid form comprising at least one component selected from the group consisting of mannitol, a monosaccharide, a disaccharide, a sweetener, a bicarbonate buffer, a phosphate buffer, a citrate buffer or citric acid, a binding agent, and a preservative.

In an aspect of the combination, the standard-of-care medicine is selected from the group consisting of an opioid antagonist, and opioid agonist, and a mixed opioid agonist/antagonist.

In an aspect of the pharmaceutical compositions of the combination, the at least one beta adrenergic receptor antagonist agent is selected from the group consisting of propranolol, atenolol, alprenolol, acebutolol, betaxolol, bisoprolol, bucindolol celiprolol, nadolol, sotalol, esmolol, carteolol, carvedilol mepindolol, nebivolol, oxprenolol, penbutolol, pindolol, landiolol, metoprolol, timolol, labetolol, and/or pharmaceutically acceptable salts thereof, and Eucommia extract, an active pharmaceutical ingredient of Eucommia, and/or pharmaceutically acceptable salt thereof.

In an aspect of the pharmaceutical compositions of the combination, the at least one antiemetic muscarinic receptor antagonist agent is selected from the group consisting of scopolamine, diphenhydramine, meclizine, buclizine, cyclizine, hydroxyzine, pirenzepine, benztropine (benzatropine), butylscopolamine, methylscopolamine, doxylamine, promethazine, trihexyphenidyl, orphenadrine and/or pharmaceutically acceptable salts thereof.

In a further aspect of the pharmaceutical compositions of the combination, the at least one beta adrenergic receptor antagonist agent and/or pharmaceutically acceptable salts thereof, is in an amount of about 10 to 100 mg/dose, and the at least one antiemetic muscarinic receptor antagonist agent and/or pharmaceutically acceptable salts thereof, is in an amount of about 0.05 to 100 mg/dose.

In an aspect of the pharmaceutical compositions of the combination, the at least one beta adrenergic receptor antagonist agent and/or pharmaceutically acceptable salts thereof, is in an amount of about 10 to 100 mg/dose, and the at least one antiemetic muscarinic receptor antagonist agent and/or pharmaceutically acceptable salts thereof, is scopolamine in an amount of about 0.05 to 1 mg/dose.

In an aspect of the pharmaceutical compositions of the combination, the at least one antiemetic muscarinic receptor antagonist agent is scopolamine, and wherein the at least one beta adrenergic receptor antagonist agent and the at least one antiemetic muscarinic receptor antagonist agent are combined in amounts therapeutically effective for pin, symptomatic treatment in a ratio which is within the range of 300:1 to 40:1 (beta adrenergic receptor antagonist agent:antiemetic muscarinic receptor antagonist agent).

In an aspect of the pharmaceutical compositions and methods of the present invention, atenolol 25 mg in combination with scopolamine HBr 0.2 mg has been prescribed and administered to patients in need of anti-anxiety treatments, and have provided evidence of a rapid-acting self-perceived calming (anxiolytic) effect [15]-[21].

Moreover, in three patients diagnosed and treated by a psychiatrist for anxiety, two patients had histories of benzodiazepine abuse [15], [17] The other patient had a history of alcohol and methamphetamine use [16]. The three patients described the compounded atenolol 25 mg—scopolamine HBr 0.2 mg medication working within about 20-30 minutes, One patient tried it sublingually with resolution of symptoms within 15 minutes. The perceived calming effects lasted 4-6 hours. Two of the patients reported their perceptions of PanX® atenolol 25 mg—scopolamine 0.2 mg as closely resembling the effects of the benzodiazepine Klonopin, albeit without using a benzodiazepine.

Furthermore, in another psychiatrist-led open label study of the same drug combination, three of the eight treated patients perceived an anxiolytic benefit mirroring that of benzodiazepines [18], In addition, Benjamin & Dooley stated, “Three of the patients in this study were being treated in the residential setting for substance abuse—patient #07 for alcohol and patients #05 and #06 for opioids” [18]. At least six of 22 patients voluntarily reported within these publications that they perceived a benzodiazepine-like calming effect of this particular drug combination when administered prn [15-19]. Thus, physicians have prescribed this atenolol—scopolamine HBr drug combination for the treatment of anxiety associated with alcohol and drug abuse and/or addiction, wherein the treatment was effective at reducing both sympathetic and parasympathetic symptoms of anxiety.
Patients, such as the at least six representatives [15-18], undergoing treatment for opioid and/or benzodiazepine substance abuse are at a high level of risk of relapse, overdoses, and overdose-related deaths, Reduction in anxiety in these patients by administration of a beta adrenergic receptor antagonist—antiemetic muscarinic receptor antagonist combination is anticipated to effectively and safely prolong abstinence and prevent overdoses, overdose deaths, and other drug or alcohol-related deaths.

### DETAILED DESCRIPTION OF THE INVENTION

The present invention relates to compositions and methods for treating subjects who have, or have had, a substance use disorder and are abstinent to substance use, the method comprising administering to the subject, a pharmaceutical composition consisting essentially of a beta adrenergic receptor antagonist drug and an antiemetic muscarinic receptor antagonist drug in a therapeutically effective amount to stop or reduce the symptoms of anxiety and/or panic in the subject incident to substance use disorder treatment and/or recovery.

Subjects or patients who benefit from treatment with the pharmaceutical compositions of the invention include patients having an SUD, alcohol and/or drug addiction, including subjects currently under the care of a physician for a substance use disorder, subjects currently being detoxified from alcohol and/or drugs or have been detoxified from alcohol and/or drugs, as well as subjects receiving standard-of-care medicines for alcohol and/or drug addiction, and recovering addicts, wherein the subjects or patients are abstinent to alcohol and/or drug use. The subjects may exhibit thoughts of using alcohol and/or drugs, cravings of alcohol and/or drugs, and temptations of using alcohol and/or drugs, and desire to remain abstinent to alcohol and/or drug use.

Addiction is defined as a treatable, chronic medical disease, People with addiction may abuse substances, like alcohol and/or drugs, and engage in behaviors that become compulsive and often continue despite harmful consequences.

A subject or patient “having alcohol and/or drug addiction” as used herein includes, for example, subjects who have, or have had a substance use disorder; subjects being treated for a substance use disorder, including subjects currently under the care of a physician for a substance use disorder; subjects currently being detoxified from alcohol and/or drugs or have been detoxified from alcohol and/or drugs; subjects under maintenance treatments and receiving standard-of-care medicines for alcohol and/or drug addiction; recovering addicts or former addicts who are not receiving standard-of-care medicines for addiction; and recovering addicts.

The Diagnostic and Statistical Manual of Mental Disorders (DSM-5), by the American Psychiatric Association characterizes “Addictive Disorders” as substance use disorders (SUDs), such as alcohol use disorder (AUD) and opioid use disorder (OUD), among others.

SUD is defined as: A problem of substance use leading to great impairment or distress, as shown by at least two of the following within 1 year: Impaired Control, Social Problems, Risky Use, and Drug Effects, Substance use disorders involve a pathologic pattern of behaviors in which subjects continue to use a substance, or abuse a substance, despite experiencing significant problems related to its use.

OUD is defined in the DSM-5 assessment criteria to be a problematic pattern of opioid use leading to clinically significant impairment or distress. Addiction to opioids is sometimes referred to as opioid dependence.

As used herein, the phrase“opiate compound” or “opioid” refers to any compound having a morphine-based ring structure such that the structure-activity relationships of the compound results in physiological binding affinity to an opiate receptor. Opiate compounds may include, for example, heroin, opium, codeine, meperidine, hydromorphone, oxycontin, hydrocodone, oxycodone, fentanyl, morphine, methadone, tramadol, and the like.

A “craving” as used herein means a state of desire to use substances or engage in addictive behaviors.

The terms “abstinent” or “abstaining from” or “a period of abstinence from” refers to the state of being without the use of substances, i.e., alcohol and/or drugs, which a subject is or has been dependent. “Abstinence” is the intentional and consistent restraint from the pathological pursuit of reward and/or relief that involves the use of substances such as alcohol and drugs, and other behaviors.

Sobriety may be defined as a state of sustained abstinence to substance use or substance abuse with a clear commitment to and active seeking of balance in the biological, psychological, social and spiritual aspects of an individual's health and wellness that were previously compromised by active addiction.

With respect to subjects who have alcohol and/or drug addiction, have been or are being treated for chemical/drug dependency, opioid dependency, and/or alcoholism, and who are abstinent of alcohol and/or drug abuse, such subjects may be “at risk for” relapse to substance use due to symptoms of anxiety or stress that produces anxiety.

As used herein, the term “relapse” (or, “recidivism”), as it relates to a subject being treated for substance use, means a tendency to return to a previous mode of substance use or substance abuse.

Relapse is process in which an individual who has established abstinence or sobriety experiences recurrence of signs and symptoms of active addiction, often including resumption of the pathological pursuit of reward and/or relief through the use of substances and other behaviors. Relapse can be triggered by exposure to substances and behaviors, by exposure to environmental cues to use, and by exposure to emotional stressors, which triggers heighten activity in brain stress circuits which may lead to anxiety and the symptoms thereof.

Anxiety and stress that produces anxiety are key factors, among others (e.g., “drug-related cues” or “drug-associated cue”), that are associated with the likelihood of relapse to substance use. These two factors (anxiety and stress) are predictive of relapse and vulnerability to addiction, and thus one can also reasonably assert that these factors increase the risk of relapse to substance use (substance abuse) and are causative in relapse in some patients.

Triggers involve feelings, thoughts or emotions formerly associated with substance abuse. Emotions that can act as internal triggers may include fear and anxiousness. When these internal triggers arise, they may cause individuals to crave substances and may fuel the temptation to revert to substance abuse. The cravings and temptations due to internal triggers, such as fear and anxiousness, increase the risk of relapse to alcohol and/or drug use in the individual.

In the context of the present invention, “treatment and/or prevention of relapse” may also be interpreted as “reducing the occurrence” for relapse to substance use or substance abuse. As used herein, the phrase “reduce the risk of relapse”, include therapies which prolong the length of time a subject remains abstinent from the use of drugs and/or alcohol to which the subject has become dependent. Ameliorating and/or reducing sensations of fear and/or anxiety reduces the risk of relapse to alcohol and/or drug use in the addicted individual.

The language “whereby the risk for relapse to substance abuse is reduced” or “thereby reducing the risk of relapse” means that, in the subject who is being treated with the pharmaceutical composition of the invention consisting essentially of a beta adrenergic receptor antagonist drug and an antiemetic muscarinic receptor antagonist drug in therapeutically effective amounts to stop or reduce the symptoms of panic and/or anxiety, the risk for relapse to substance abuse is reduced compared to a subject who is being treated for alcohol and/or drug addiction and who is not administered an anxiolytic medication.

The pharmaceutical compositions of the present invention alleviate anxiety symptoms (e.g., fear, anxiousness, avoidance, tachycardia, etc.), which are understood by those skilled in the art to increase the likelihood of relapse to substance use, or substance abuse, and reduce the occurrence of relapse to substance abuse, for example, alcohol and/or drugs. The methods of the present invention comprise administering to subject a pharmaceutical composition consisting essentially of a beta adrenergic receptor antagonist drug and an antiemetic muscarinic receptor antagonist drug in therapeutically effective amounts to stop or reduce the symptoms of panic or anxiety, and thereby reduce the risk for relapse to substance abuse.

The autonomic nervous system (ANS) plays essential roles in processing stress and anxiety, and chronic substance abuse can result in dysregulation of the ANS [4]. The pharmaceutical compositions of the invention consisting essentially of a therapeutically effective amount of at least one beta adrenergic receptor antagonist agent and/or pharmaceutically acceptable salts thereof, and at least one antiemetic muscarinic receptor antagonist agent and/or pharmaceutically acceptable salts thereof, are an anxiolytic that affects both the sympathetic and parasympathetic symptoms of the ANS, and can treat anxiety symptoms in patients affected by anxiety disorders or situational anxiety. Therefore, it follows that a pharmaceutical composition consisting essentially of a therapeutically effective amount of at least one beta adrenergic receptor antagonist agent and/or pharmaceutically acceptable salts thereof, and at least one antiemetic muscarinic receptor antagonist agent and/or pharmaceutically acceptable salts thereof, when administered pro re nata (p.r.n., “as needed”), to patients affected by SUDs, will result in a rapid-onset anxiolytic treatment benefit. In fact the anxiolytic benefit has already been observed in 22 patients in physician-sponsored studies administering a pharmaceutical composition consisting essentially of a therapeutically effective amount of atenolol and scopolamine [15]-[21].

It follows that with continued p.r.n. administration (either occasional or daily use) this anxiolytic effect over days, weeks, months, and years of the use of a pharmaceutical composition consisting essentially of a therapeutically effective amount of at least one beta adrenergic receptor antagonist agent and/or pharmaceutically acceptable salts thereof, and at least one antiemetic muscarinic receptor antagonist agent and/or pharmaceutically acceptable salts thereof, may also positively impact the risk of relapse to alcohol and/or drug use. In other words, if anxiety and stress that produces anxiety are key factors that can psychologically and physiologically “drive” an abstinent addict to return to drugs or alcohol, then a pharmaceutical composition consisting essentially of a therapeutically effective amount of at least one beta adrenergic receptor antagonist agent and/or pharmaceutically acceptable salts thereof, and at least one antiemetic muscarinic receptor antagonist agent and/or pharmaceutically acceptable salts thereof, administered over time can also abrogate this relapse, providing a prophylactic benefit to the patients. Hence, the pharmaceutical compositions of the invention consisting essentially of a therapeutically effective amount of at least one beta adrenergic receptor antagonist agent and/or pharmaceutically acceptable salts thereof, and at least one antiemetic muscarinic receptor antagonist agent and/or pharmaceutically acceptable salts thereof serve as a p.r.n. anxiolytic treatment per se, and may be used prophylactically over time thereby reducing the risk of relapse to alcohol and/or drug use in patients.

Opioid deterrent standard-of-care medicines are known in the art of OUD treatment and may be selected from methadone (opioid agonist), buprenorphine (opioid agonist or opioid partial agonist), buprenorphine in combination with naloxone (opioid antagonist), and naltrexone (opioid antagonist), and pharmaceutically acceptable salts thereof.

Standard-of-care medicines for the treatment of alcohol dependence or OUD are selected from disulfiram (alcohol abuse-deterrant agent that produces unpleasant effects), acamprosate (to stabilize neurochemistry), and naltrexone (opioid antagonist).

The invention provides methods for treating symptoms of anxiety and/or panic in a subject who is receiving therapy for alcohol and/or drug addiction comprising administering a pharmaceutical composition consisting essentially of a beta adrenergic receptor antagonist drug and an antiemetic muscarinic receptor antagonist drug in a therapeutically effective amount to stop or reduce the symptoms of anxiety and/or panic in a subject who is undergoing treatment for drug addiction and/or a subject who has been detoxified as part of a treatment for alcohol and/or drug addiction and is compliant with a deterrent standard-of-care medicine.

The invention provides methods for treating symptoms of anxiety and/or panic in a subject who is receiving therapy for alcohol and/or drug addiction comprising administering a combination of a standard-of-care medicine and a pharmaceutical composition consisting essentially of a beta adrenergic receptor antagonist drug and an antiemetic muscarinic receptor antagonist drug in a therapeutically effective amounts for p.r.n. treatment to stop or reduce the symptoms of anxiety and/or panic in a subject who is undergoing treatment for alcohol and/or drug addiction.

The administration of the pharmaceutical composition consisting essentially of a beta adrenergic receptor antagonist drug and an antiemetic muscarinic receptor antagonist drug may be done before, or after, the administration of a standard-of-care medicine, or may be administered coincidentally, that is, at the same time.

The term “therapeutically effective” applied to dose or amount refers to that quantity of a compound or pharmaceutical composition that is sufficient to result in a desired activity upon administration to a living animal body. A therapeutically effective amount can be administered in one or more administrations, applications, or dosages, Determination of an effective amount for a given administration is well within the ordinary skill in the pharmaceutical arts.

The term “anxiety” covers a vast breadth of definitions and symptoms. The present invention addresses all or most of the symptoms of acute anxiety or panic episodes, neither of which is addressed by a beta blocker alone or an antiemetic muscarinic receptor antagonist agent alone. The present invention can achieve desirable pharmacologic effects upon a diverse array of cardiovascular and non-cardiovascular symptoms at appropriate doses of APIs that are antiemetic, fast-acting, and coincidentally without the use of addictive drugs and/or dependent drugs, and/or Controlled Substances as APIs. Furthermore, augmentation of a beta blocker with an antiemetic agent that only affects the symptoms of nausea, vomiting, and/or motion sickness per se provides anxiolytic superiority over a beta blocker alone or an antiemetic agent alone.

The phases “treatment and/or prevention of panic and/or anxiety” and “treatment and/or prevention of the symptoms of anxiety” include reducing the length of time a subject exhibits the symptoms of anxiety, abrogating anxiety and the symptoms of anxiety, reducing/alleviating the symptoms of anxiety, protecting the subject from experiencing anxiety and the symptoms of anxiety.

In an embodiment, this invention provides a method for reducing the risk of relapse in a subject who is undergoing treatment for alcohol and/or drug addiction and is abstinent to alcohol and/or drug use comprising administering to the subject anticipating panic or anxiety and/or at the time of a trigger circumstance for panic or anxiety or a drug-associated cue, a pharmaceutical composition consisting essentially of a beta adrenergic receptor antagonist drug and an antiemetic muscarinic receptor antagonist drug in a therapeutically effective amount to stop or reduce the symptoms of panic or anxiety, whereby the risk for relapse to substance abuse is reduced in the subject.

In a further embodiment, this invention provides a method for reducing the risk of relapse in a subject who is undergoing treatment for alcohol and/or drug addiction and is abstinent to alcohol and/or drug use comprising administering to a subject who is undergoing treatment for alcohol and/or drug addiction and is abstinent to alcohol and/or drug use, a pharmaceutical composition consisting essentially of a beta adrenergic receptor antagonist drug and an antiemetic muscarinic receptor antagonist drug in a therapeutically effective amount to stop or reduce the symptoms of anxiety or panic at the onset of anxiety or panic, whereby the risk for relapse to substance abuse is reduced in the subject.

In yet another embodiment, this invention provides a method for treating panic and/or anxiety, and the symptoms thereof, in a subject comprising concomitantly administering to the subject, a standard-of-care medicine for alcohol and/or drug addiction and a pharmaceutical composition consisting essentially of a beta adrenergic receptor antagonist agent and an antiemetic muscarinic receptor antagonist agent in a therapeutically effective amount to stop or reduce the symptoms of anxiety or panic, whereby the risk for relapse to substance abuse is reduced in the subject.

The pharmaceutical compositions of the invention consisting essentially of a therapeutically effective amount of at least one beta adrenergic receptor antagonist agent and/or pharmaceutically acceptable salts thereof, and at least one antiemetic muscarinic receptor antagonist agent and/or pharmaceutically acceptable salts thereof, (e.g., atenolol and scopolamine, propranolol and scopolamine, atenolol and promethazine, etc.) provide complementary pharmacologic benefits by interaction of the drug actives with two or more dissimilar molecular targets.

The present invention stops or reduces (i.e., minimize the number or severity of) symptoms associated with panic and anxiety incident to SUD treatment and/or recovery.

The active pharmaceutical ingredients (APIs) of the pharmaceutical compositions of the invention have desirable historic performance characteristics in humans. For example, APIs can be selected that are non-addicting and/or either non-sedating or minimally sedating. Beta blockers are non-addicting and non-sedating. The antiemetic muscarinic receptor antagonist APIs are non-addicting, and can be non-sedating or minimally sedating at antiemetic doses. Furthermore, both classes of these APs have been demonstrated to be well tolerated with minimal side effects during at least five decades of human use.

Given the episodic nature of panic and acute anxiety incident to SUD treatment and/or recovery, it is beneficial to have a suitable pharmaceutical composition of the present invention available to a subject/patient with a substance use disorder who desires relief from symptoms of panic and/or anxiety and does not wish to be treated with an addictive substance, for example, a benzodiazepine. The pharmaceutical composition could be in a purse, a pocket, a home, a workplace, yet could be immediately available for use “as needed” (p.r.n.) on occasion. At the time a patient encounters or anticipates an episode (e.g., a “trigger” circumstance was likely to occur) the pharmaceutical compositions of the invention consisting essentially of a therapeutically effective amount of at least one beta adrenergic receptor antagonist agent and/or pharmaceutically acceptable salts thereof, and at least one antiemetic muscarinic receptor antagonist agent and/or pharmaceutically acceptable salts thereof, can be administered mucosally or orally to stop or reduce (i.e., minimize the number or severity of) the symptoms of panic or anxiety.

The pharmaceutical compositions of the invention can replace conventional oral anxiolytic medications in subjects who have drug and/or alcohol dependency, and could reduce addiction, dependence, sedation, lethargy, and drug tolerance to the conventional anxiolytics, such as benzodiazepines.

The present invention solves the problem of alleviating symptoms of anxiety in patients being treated for alcohol and/or drug addiction without the use of benzodiazepines. None of the active pharmaceutical ingredients (APIs) of the present invention are “Controlled Substances” as classified by the U.S. FDA Schedules 1-4.

Furthermore, replacement of a “Controlled Substance” (i.e., benzodiazepine) with effective and non-addicting APIs of the pharmaceutical compositions of the invention is advantageous, particularly in subjects who may be predisposed to addiction, as well as subjects who are undergoing treatment for SUD, including alcohol and/or drug addiction, including opioid addiction, and/or subjects who are recovering from alcohol and/or drug addiction.

In an embodiment, the mucosal route of delivery provides for rapid penetration through the mucosa, followed by uptake into the circulatory system, resulting in systemic bioavailability without first-pass hepatic metabolism, Rapid drug delivery is feasible for mucosal routes, such as the sublingual, buccal, nasal, vaginal, or rectal routes. The speed at which one would expect a therapeutic benefit by a mucosal route would be second only to the intravenous (parenteral) route of administration. Oral or transdermal delivery is expected to be slower than the mucosal route, by minutes if not hours. Thus, in general the relative rates of absorption and therapeutic effect would be: intravenous>mucosal>oral>transdermal. Of these options, mucosal delivery is “optimal” and preferred in “time is of the essence” therapy, because it is convenient to administer and does not involve the self-administration by injection with needles.

The present invention provides a new paradigm for pharmaceutical therapy for patients being treated for a substance use disorder who have episodes of panic, fear and/or anxiety and who desire to abstain from addictive substances. The present invention may be an episodic therapy for the symptoms of panic or anxiety, per se, incident to SUD treatment and recovery, and is to be taken “as needed” at the discretion of the patient.

In an embodiment, a pharmaceutical composition consisting essentially of an antiemetic muscarinic receptor antagonist agent and a beta adrenergic receptor antagonist agent is self-administered by a subject pro re nata, that is, “as needed” by the subject to provide an anxiolytic benefit and to reduce the risk of relapse to substance abuse. The subject may self-administer the pharmaceutical composition occasionally as needed, for example, when the patient is experiencing or is anticipating experiencing the symptoms of anxiety and/or panic. The subject may self-administer a pharmaceutical composition consisting essentially of an antiemetic muscarinic receptor antagonist agent and a beta adrenergic receptor antagonist agent as needed in anticipation of, or when encountering a trigger circumstance for alcohol and/or drug use. The subject may self-administer the pharmaceutical composition consisting essentially of an antiemetic muscarinic receptor antagonist agent and a beta adrenergic receptor antagonist agent as needed to prevent and/or reduce the symptoms of anxiety and panic, which self-administration includes, monthly, weekly, daily, multiple times a day, and less than once daily administration, at the discretion of the subject depending on their need for an anxiolytic medication.

In an embodiment, the present invention provides “fast-acting” drugs for the p.r.n. treatment of the acute symptoms of a panic attack and anxiety per se at the time of an event or in immediate anticipation of an event.

The pharmaceutical compositions of the present invention consist essentially of an antiemetic muscarinic receptor antagonist agent and a beta adrenergic receptor antagonist agent, wherein the antiemetic muscarinic receptor antagonist drug and/or beta adrenergic receptor antagonist drug may be in the form of a free base or a pharmaceutically acceptable salt thereof.

The phrase “pharmaceutically acceptable”, as used in connection with compositions of the invention, refers to molecular entities and other ingredients of such compositions that are physiologically tolerable and do not typically produce untoward reactions when administered to a mammal (e.g., human). Preferably, as used herein, the term “pharmaceutically acceptable” means approved by a regulatory agency of the Federal or a state government or listed in the U.S. Pharmacopeia (USP), National Formulary (NF), or other generally recognized pharmacopeia for use in mammals, and more particularly in humans, APIs of the present invention may be in the form of pharmaceutically acceptable salts. “Pharmaceutically acceptable salts” refers to those salts which possess the biological effectiveness and properties of the parent compound and which are not biologically or otherwise undesirable.

The pharmaceutical compositions of the present invention comprise a beta adrenergic receptor antagonist agent selected from the group consisting of propranolol, atenolol, alprenolol, acebutolol, betaxolol, bisoprolol, bucindolol, celiprolol, nadolol, sotalol, esmolol carteolol, carvedilol, mepindolol, nebivolol, oxprenolol, penbutolol, pindolol landiolol, metoprolol, timolol, labetolol, and Eucommia extract or an active pharmaceutical ingredient of Eucommia, wherein the beta adrenergic receptor antagonist agent may be in the form of a free base or a pharmaceutically acceptable salt thereof. In addition, one can envision a medicinal chemistry approach to develop novel structure-activity relationship (SAR) series of compounds related to beta blockers and/or their active metabolites in vivo, which could be beneficial APIs for compositions of the present invention.

The pharmaceutical compositions of the present invention may include, specifically, propranolol or atenolol (or other beta blockers) and scopolamine (or other muscarinic receptor antagonist agents), or the salts, pro-drugs, or analogs, derivatives, or metabolites thereof, which may be administered to a subject being treated for alcohol and/or drug addiction.

Propranolol is the most thoroughly studied of the beta blockers, and serves as the prototype for this class of drugs. Propranolol is nonselective, whereas atenolol is a beta-1 selective beta blocker. Atenolol is one of the preferred beta blockers of the pharmaceutical compositions, and is considered preferable over propranolol with regard to receptor selectivity and a lack of an unpleasant taste and mouth paresthesia, especially so if intended for mucosal absorption within the mouth.

With regard to the pharmaceutically acceptable salts, propranolol HCl may be administered in a target dose in the range of about 10-100 mg per dose of propranolol in adults. It may be preferable to use a target dose of about 20-80 mg per adult. It might be even more preferable to use a target dose of about 20-40 mg per adult, especially for mucosal delivery. A specific dose naturally varies depending on the dosage procedure, the conditions of a patient or a subject such as age, body weight, sex, sensitivity, dosage period, drugs used in combination, seriousness of the disease. The appropriate dose and dosage times under certain conditions can be determined based on the above-described indices but may be refined and ultimately decided according to the judgment of the practitioner and each patient's circumstances (age, general condition, severity of symptoms, sex, etc.) according to standard clinical techniques. Given that propranolol produces mouth paresthesia and an adverse taste, oral administration is preferred for this beta blocker.

The present invention provides a target dose of atenolol which is administered in the range of about 10-100 mg per dose in adults. It may be preferable to use a target dose of about 20-80 mg per adult. It may be even more preferable to use a target dose of about 20-50 mg per adult, especially for mucosal delivery. A specific dose naturally varies depending on the dosage procedure, the conditions of a patient or a subject such as age, body weight, sex, sensitivity, dosage period, drugs used in combination, seriousness of the disease. The appropriate dose and dosage times under certain conditions can be determined based on the above-described indices but may be refined and ultimately decided according to the judgment of the practitioner and each patient's circumstances (age, general condition, severity of symptoms, sex, etc.) according to standard clinical techniques.

The pharmaceutical compositions of the present invention comprise an antiemetic muscarinic receptor antagonist agent selected from the group consisting of scopolamine, diphenhydramine, meclizine, buclizine, cyclizine, hydroxyzine, pirenzepine, benztropine (benzatropine), butylscopolamine, methylscopolamine, doxylamine, promethazine, trihexyphenidyl, and orphenadrine (or its metabolite, tofenacine), wherein the antiemetic muscarinic receptor antagonist agent may be in the form of a free base or a pharmaceutically acceptable salt thereof.

Scopolamine (or a pharmaceutically acceptable salt thereof) is an antiemetic antagonist of muscarinic acetylcholine receptors. It is a nonselective muscarinic receptor inhibitor that can inhibit all five human receptor subtypes with 0.34-53 nM Ki values. It is lipophilic and crosses the blood-brain barrier to exert psychic (NS) pharmacologic effects, Scopolamine can also be absorbed mucosally, as demonstrated by sublingual delivery.

With regard to the pharmaceutically acceptable salts, scopolamine HBr (scopolamine hydrobromide) may be administered a target dose range of about 0.05-1.0 mg of scopolamine in adults. It might be preferable to use a target dose of 0.2-0.6 mg per adult for oral delivery, and 0.05-0.3 mg per adult for mucosal delivery. The dose for adolescents and children would be less than an adult's dose. A specific dose naturally varies depending on the dosage procedure, the conditions of a patient or a subject such as age, body weight, sex, sensitivity, dosage period, drugs used in combination, seriousness of the disease. The appropriate dose and dosage times under certain conditions can be determined based on the above-described indices but may be refined and ultimately decided according to the judgment of the practitioner and each patient's circumstances (age, general condition, severity of symptoms, sex, etc.) according to standard clinical techniques.

Closely-related derivatives of scopolamine are alternative antiemetic muscarinic receptor antagonist APIs, such as butylscopolamine, methylscopolamine, and benztropine (benzatropine). Thus, in an embodiment, central-acting lipophilic antiemetic muscarinic receptor antagonist agents (e.g., scopolamine) are preferred. In addition, one can envision a medicinal chemistry approach to develop novel structure-activity relationship (SAR) series of CNS-active compounds related to scopolamine or other muscarinic receptor inhibitors, which could be beneficial APIs for compositions of the present invention.

In addition to the scopolamine family of APIs, other closely-related families of APIs exhibiting antimuscarinic activities are included in the present invention. In some cases the APs also exhibit antihistamine properties. Examples of other antiemetic muscarinic receptor antagonist agents include: (a) diphenhydramine, orphenadrine and its metabolite tofenacine, and doxylamine, all based upon an ethanolamine moiety; and (b) meclizine, buclizine, cyclizine, hydroxyzine, and pirenzepine, all based upon a piperazine moiety. Hydroxyzine is sometimes prescribed for anxiety and might be beneficial in treating panic disorder. As stated above, CNS accessibility by the muscarinic receptor antagonist is preferable, Pirenzepine is M1 selective, but it lacks CNS effects, and is thus less likely to produce some of the advantageous properties of this class of antagonists. Furthermore, other types of antiemetic antimuscarinic agents are available, such as promethazine and trihexyphenidyl.

Furthermore, in view of muscarinic receptor binding affinities scopolamine, promethazine, and diphenhydramine are preferred API embodiments.

The pharmaceutical compositions of the present invention may comprise one or more excipients, Excipients which may be used include carriers, surface active agents (surfactants), thickening (viscosity) agents, emulsifying agents, binding agents, gelatin, dispersion or suspension agents, buffering agents, penetration-enhancing agents, solubilizers, colorants, sweeteners, flavoring agents, coatings, disintegrating agents, lubricants, preservatives, isotonic agents, and combinations thereof. The selection and use of suitable excipients is taught in Gennaro, ed., Remington: The Science and Practice of Pharmacy, 20th Ed. (Lippincott Williams & Wilkins 2003), the disclosure of which is incorporated herein by reference.

The term “carrier” applied to pharmaceutical compositions of the invention refers to a diluent, excipient, or vehicle with which an active compound is administered. Such pharmaceutical carriers can be liquids, such as water, saline solutions, aqueous dextrose solutions, aqueous glycerol solutions, and lipids and oils, including those of petroleum, animal vegetable or synthetic origin. Suitable pharmaceutical carriers are described in “Remington's Pharmaceutical Sciences” by E.W. Martin, 18^{th} Edition.

The pharmaceutical compositions of the present invention consisting essentially of beta adrenergic receptor antagonists and antiemetic muscarinic receptor antagonists may be in the form of a solid, a semi-solid, or a liquid dose form. Examples include sprays, elixirs, solutions, suspensions, emulsions, gels, creams, gums, powders, tablets, capsules, troches, suppositories, pills, and films, A variety of mechanical devices may be used to dispense the formulations, for instance pump applicators, spray applicators, and compressible tube dispensers. The devices may deliver calibrated unit doses, for instance in the cases of liquid or semi-solid dosage forms.

The pharmaceutical compositions of the present invention, when intended for sublingual, buccal, and/or oral delivery, may benefit from taste-masking with natural and/or artificial flavors and/or sweeteners (e.g., mannitol, sucralose, monosaccharides, and disaccharides). Mouth paresthesia (numbness) can occur with propranolol HCl as a perceived adverse effect. Paresthesia and an undesirable taste were observed for sublingual dose forms containing propranolol during development. However, neither property was observed for atenolol in sublingual dose forms, thus providing two distinct advantages for this particular beta blocker.

In addition, using methods known to those skilled in the art, analogs and derivatives of the APIs of the invention can be created which have improved therapeutic efficacy in controlling panic and anxiety, i.e., higher potency and/or selectivity at a specific targeted receptor type, either greater or lower ability to penetrate mammalian blood-brain barriers, fewer side effects, etc.

The term “analog” or “derivative” or “metabolite” is used herein in the conventional pharmaceutical sense, to refer to a molecule that structurally resembles a reference molecule, but has been modified in a targeted and controlled manner to replace one or more specific substituents of the reference molecule with an alternate substituent, thereby generating a molecule which is structurally similar to the reference molecule. Synthesis and screening of analogs (e.g. using structural and/or biochemical analysis), to identify slightly modified versions of a known compound which may have improved or biased traits (such as higher potency and/or selectivity at a specific targeted receptor type, greater ability to penetrate mammalian blood-brain barriers, fewer side effects, etc.) is a drug design approach that is well known in pharmaceutical chemistry.

The pharmaceutical compositions of the present invention may include biocompatible buffering agents, such as sodium bicarbonate (pKa 0.4), sodium phosphate buffer (mono- and di-basic admixtures; pKa 6.8), or citrate buffer or citric acid. For instance, in the event of sublingual or buccal delivery buffering to approximate the pH of saliva (pH 62-7.4) might be advantageous. In certain instances the chemical stability of one or more of the APIs within the formulation might benefit from the inclusion of biocompatible buffering agents. In certain conditions the physical stability of the liquid and/or semi-solid formulations might benefit from the inclusion of biocompatible buffering agents. Bicarbonates and phosphates are considered as physiologic buffers in mammals and humans. In certain compositions it might be preferable to maintain an acidic pH or acidic-to-neutral pH to prevent decomposition of an API (e.g., propranolol).

The pharmaceutical compositions of the present invention may include chemical preservatives, in addition to ethanol and/or glycerol in liquid and semi-solid compositions. Ethanol and glycerol are known to exhibit antibacterial properties. Examples of commonly used preservatives include sodium benzoate, benzyl alcohol, methyl paraben, propyl paraben, and butyl paraben, among others.

The pharmaceutical compositions of the present invention, when in liquid or semi-solid forms, may include one or more penetration-enhancing solvents such as ethanol, glycerol, propylene glycol and/or ethoxydiglycol. In some circumstances, dimethylsulfoxide (DMSO) may be included as a skin penetration enhancer. However, DMSO may yield an unpleasant garlic flavor, even when applied to sites remote from the oral cavity.

Compositions of the present invention can also be formulated for vaginal or rectal administration, such as suppositories, films, viscous gels, creams, or retention enemas (e.g., containing conventional suppository bases such as cocoa butter or other glycerides).

The compositions may, if desired, be presented in a pack or dispenser device which may contain one or more unit dosage forms containing the active ingredient(s). The pack may, for example, comprise metal or plastic foil, such as a blister pack. Compositions of the invention formulated in a compatible pharmaceutical carrier may also be prepared, placed in an appropriate container, and labeled for treatment of an indicated condition.

The pharmaceutical compositions of the present invention may include surfactants, such as phospholipids (e.g., lecithin) and sodium lauryl sulfate Surfactants help solubilize otherwise insoluble APIs or ingredients. Surfactants might be needed in certain drug combination compositions to produce emulsions or suspensions.

The pharmaceutical compositions of the present invention may include hydrophobic components, such as lipids, oils (e.g., isopropyl myristate and isopropyl palmitate), fatty acids, unsaturated fatty acids, waxes, petrolatum, lanolin, etc.

The pharmaceutical compositions of the present invention may include manufactured admixture “bases” typically used in extemporaneous compounding of pharmaceutical products. These “bases” are commercially available to pharmacists working in compounding pharmacies. In this context, the term “base” does not refer to alkalinity or pH. Rather they are “bases” into which active and/or inactive ingredients are mixed. The “bases” can be intended for specific routes of delivery, such as elixirs, syrups, and sweetened syrups for oral and oral mucosal delivery.

For administration in liquid or semi-solid forms, the APIs can be combined with pharmaceutically acceptable carriers (e.g., ethanol, glycerol, water), suspending agents (e.g. sorbitol syrup), emulsifying agents (e.g., lecithin), viscosity agents (e.g., methyl cellulose, polyethylene glycols, or Carbomer Homopolymer Type A), non-aqueous vehicles (e.g, a plant-derived oil), preservatives (e.g., a paraben), and the like. Stabilizing agents such as antioxidants (e.g., citric acid) can also be added.

For administration in the form of a tablet (sublingual or oral) or capsule (oral), the APIs can be combined with pharmaceutically acceptable excipients, such as binding agents (e.g., starch) fillers (e.g., mannitol); lubricants (e.g, magnesium stearate); dispersants or disintegrants (e.g, starch); or wetting agents (e.g., sodium lauryl sulphate), coloring and flavoring agents, gelatin, collagen, sweeteners (e.g., mono- or di-saccharides, sucralose), natural and synthetic gums (e.g, acacia), buffer salts (e.g., sodium bicarbonate), waxes, and the like.

The tablets can be coated by methods well known in the art. The compositions of the invention can be also introduced in microspheres, e.g., fabricated from polyglycolic acid/lactic acid (PGLA). Liquid preparations for oral administration can take the form of, for example, solutions, syrups, emulsions or suspensions, or they can be presented as a dry product (e.g., powder) for reconstitution with water or other suitable vehicle before use. Preparations for oral administration can be suitably formulated to give controlled or postponed release of the active compound, Controlled or postponed release may apply to one or more of the APIs within the composition, and may also apply to portions of one or more of the APIs within the composition. The APIs can also be administered in the form of liposome delivery systems. Liposomes can be formed from a variety of lipids and phospholipids, such as cholesterol, stearylamine or phosphatidylcholines, as is well known.

For administration by nasal inhalation, the therapeutics according to the present invention can be conveniently delivered in the form of an aerosol spray presentation from pressurized packs or a nebulizer, with the use of a suitable propellant, (e.g, dichlorodifluoromethane). In the case of a pressurized aerosol, the dosage unit can be determined by providing a valve to deliver a metered amount, Capsules (e.g., gelatin) and cartridges for use in an inhaler or insufflator can be formulated containing a powder mix of the compound and a suitable powder base such as lactose or starch.

The pharmaceutical compositions of the present invention may be delivered by mucosal or oral routes. A preferred route of delivery for the present invention is mucosal, as this route avoids GI metabolism and more significantly first-pass metabolism by the liver. For instance, within the oral mucosal environment at least two routes of absorption are possible—sublingual and buccal. Mucosal delivery is beneficial and preferred for p.r.n, administration, as well as to hasten the effects of the compositions. Orally disintegrating tablets that disperse rapidly (e.g., within 10 seconds or less) are a preferred pharmaceutical composition for intra-oral mucosal delivery. Alternatively, a “chewing” gum (for mucosal sublingual, buccal, and/or oral delivery) might have the additional perceived benefit as a palliative agent A patient could chew on it for some time while anticipating or waiting for the onset of a pharmacologic benefit. Intra-oral dispersion can result in drug absorption mucosally (pre-gastric) and/or orally (gastric), depending on the pharmaceutical composition, chemical properties of the APIs, resident time in the intra-oral compartment, among other factors.

Outside of the oral cavity, the pharmaceutical compositions of the present invention could be delivered to the mucosa of the rectum, the vagina, or nasal passages. These latter routes could be used in certain circumstances, such as a patient's aversion to the taste of a composition, or a patient's unwillingness or inability to swallow, or while a patient is prone to vomiting.

In addition to various mucosal routes of delivery (with expected rapid bioavailability), another preferred route is oral, essentially for ease-of-use by the patient. The oral route (e.g., tablets, capsules, and elixirs) is preferred when a patient anticipates in the future (e.g, in one hour) a“trigger” circumstance that is likely to lead into the onset of symptoms of panic or anxiety. The patient who expects to encounter a trigger circumstance self-administers the medication p.r.n. orally and waits a sufficient period of time (e.g., one hour) to enable GI absorption and bioavailability, prior to encountering the trigger. In other words, the effect of the oral route is likely to be somewhat delayed relative to a mucosal route.

In an embodiment, a topical formulation might be preferred, for instance to provide slower release or sustained-release of the combination of APIs. In another embodiment, in certain circumstances a parenteral formulation might be preferred, such as an intravenous injection intended for very rapid effect (e.g, less than 15 minutes) during symptoms of panic or anxiety.

The present invention is administered as a therapy to be taken at the time of an episode or in anticipation of an episode, as a daily and/or persistent oral product “as needed” by the subject for the suppression of panic, fear and/or anxiety in subjects being treated for alcohol and/or drug addiction and are abstinent to alcohol and/or drug use, whereby the administration provides an anxiolytic effect in the subject and reduces the risk of relapse to alcohol and/or drug use. The patient is able to self-medicate at the time of symptoms, or earlier at the time of a trigger, or even earlier in anticipation of a trigger. For instance, the p.r.n. self-administration of the pharmaceutical compositions of the invention might occur 10, 20, 30, 40, 50, or 60 minutes prior to the anticipated symptoms of panic or acute anxiety. Furthermore, when “time is of the essence” the mucosal routes of delivery are preferred, especially oral mucosal (e.g., sublingual) dose forms.

In an embodiment, a sublingual or buccal formulation is administered by the patient at the time of a panic or anxiety episode or in anticipation of a trigger of an episode, thus helping enable the patient to regain some or full control over their symptoms as needed, Control over the symptoms of anxiety reduces the urge to revert to alcohol and/or drug use.

The amount of APs which can be combined with a carrier material to produce a single dosage form will vary depending upon the subject being treated, and the particular mode of administration, and will generally be that amount of the composition which produces a therapeutic effect. The dosage amount would be less in adolescents and children than in an adult, and might be a function of body mass or body surface area.

Toxicity and therapeutic efficacy of the APIs either alone or in compositions of the present invention can be determined by standard pharmaceutical procedures in experimental animals, e.g. by determining the LD₅₀ (the dose lethal to 50% of the population) and the ED₅₀ (the dose therapeutically effective in 50% of the population). The dose ratio between therapeutic and toxic effects is the therapeutic index and it can be expressed as the ratio ED₅₀/LD₅₀. APIs and/or compositions that exhibit large therapeutic indices are preferred.

specific dose of the pharmaceutical compositions of the invention varies depending on the dosage procedure, the conditions of a patient or a subject such as age, body weight, sex, sensitivity, dosage period, drugs used in combination, seriousness of the disease. The appropriate dose and dosage times under certain conditions can be determined based on the above-described indices but may be refined and ultimately decided according to the judgment of the practitioner and each patient's circumstances (age, general condition, severity of symptoms, sex, etc.) according to standard clinical techniques.

Specific embodiments disclosed herein may be further limited in the claims using “consisting of” or “consisting essentially of language”. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s) Embodiments of the invention so claimed are inherently or expressly described and enabled herein.

As used herein, the term “comprising” or “comprises” is intended to mean that the compositions and methods include the recited elements, but not excluding others.

As used herein, the terms “optional” or “optionally” mean that the subsequently described event or condition may need not occur, and that the description includes instances where the event or condition occurs and instances in which it does not.

As used herein, the phrase “oral administration” or “orally” refers to the introduction of a pharmaceutical composition into a subject by way of the oral cavity (e.g., in aqueous liquid or solid form).

As used herein, the term “patient” or “subject” refers to a warm-blooded animal, including a human.

### EXAMPLES

The subject matter of the present invention will be better understood in connection with the following examples, which are intended as an illustration of and not a limitation upon the scope of the invention. It will be apparent to those skilled in the art that the described examples are merely representative in nature.

The pharmaceutical compositions of the present invention, together with one or more conventional adjuvants, carriers, or diluents, may be placed into the form of unit dosages, and in such form may be employed as solids (e.g., coated or uncoated tablets or filled capsules), or liquids or semi-solids (e.g., solutions, suspensions, emulsions, creams, gels, elixirs, or capsules filled with the same), all for oral or mucosal use; in the form of suppositories or capsules for rectal and vaginal administration.

Such pharmaceutical compositions and unit dosage forms thereof may comprise conventional or new ingredients in conventional or special proportions, with or without additional active compounds or principles, and such unit dosage forms may contain any suitable effective amount of the active ingredient commensurate with the intended dosage range to be employed. Tablets containing twenty (20) to two hundred (200) milligrams of active ingredient(s) or, more broadly, ten (10) to three hundred (300) milligrams of active ingredient(s) per tablet, are accordingly suitable representative unit dosage forms, as solids, With regard to liquids containing the combination therapeutic agents, such as alcoholic elixirs for oral or sublingual administration, a volume of about one hundred (100) microliters to about five (5) milliliters per unit dose is a suitable representative unit dosage volume, With regard to semi-solids containing the combination therapeutic agents, such as gels or creams for vaginal or rectal administration, a volume of about one half (0.5) milliliter to about five (5) milliliters is a suitable representative unit dosage volume. Consideration would also be given to the estimated maximum number of consecutive unit doses administered per period of time, such as per 6, 12, or 24 hours.

### Example 1. Liquid Pharmaceutical Compositions

| | | | |
|---|---|---|---|
| Ingredients: | # 1 | # 2 | # 4 |

| | | | | | | |
|---|---|---|---|---|---|---|
| Propranolol HCl | 1.2 | g | 1.2 | g | 1.2 | g |
| Scopolamine HBr, Trihydrate | 15 | mg | 30 | mg | 15 | mg |
| Ethanol, 95% | 1.5 | ml | 3.0 | ml | 3.0 | ml |
| Glycerol | 1.5 | ml | 3.0 | ml | 3.0 | ml |

| | | | | | |
|---|---|---|---|---|---|
| Propylene Glycol | 3.0 | ml | 3.0 | ml | — |

| | | | |
|---|---|---|---|
| Purified Water | QS 15 g | QS 15 g | QS 15 g |

As examples of aqueous liquid pharmaceutical compositions, alcoholic elixirs are prepared consisting of 80 mg/m propranolol HCl, 1-2 mg/ml scopolamine HBr, 10-20% ethanol, 10-20% glycerol, and 0-20% propylene glycol. The dose is 0.5 mi of the alcoholic elixir in an adult, with a delivered dose of 40 mg propranolol HCl and 0.5 or 1.0 mg scopolamine HBr. The APIs are soluble in these elixirs at room temperature, but a precipitate may form at or near freezing when stored for multiple days in a refrigerator.

The preferred route of delivery for elixir compositions is oral Taste-masking flavors and sweeteners may be incorporated into the composition, because propranolol HCl has an “acidic” and “medicine” taste and it produces mouth paresthesia when placed under or on the tongue. These undesirable properties were noted in humans during development of the liquid compositions. Alternative beta blockers may be chosen that lack these undesirable properties, such as atenolol.

The pH of the alcoholic elixirs (samples #1, 2, and 4) is ˜4.5 without buffering. It may be beneficial to include a buffering agent or agents (e.g., bicarbonate or phosphate) to increase and buffer the pH to that of the oral cavity (˜6.2-7.4) for sublingual or oral delivery. For instance, sodium bicarbonate can be included at a ratio (by mass) relative to propranolol HCl of about 1:50 to about 1:20, to increase the pH toward neutrality. For instance a ratio of ˜1:26 of sodium bicarbonate to propranolol HC raised the pH of elixir #4 to ˜pH 6.5-7.0.

The alcohols (i.e., ethanol, glycerol, and propylene glycol) within the elixir may provide penetration enhancement of the APIs and a preservative property, The elixir can be dispensed by a unit-dose metered pump or spray applicator (e.g., delivering 0.5 or 0.25 ml for each pump action).

The alcoholic elixirs (samples #1, 2, and 4) had low viscosities of <10 cP when measured using a Brookfield viscometer.

In other liquid compositions, one may substitute other beta adrenergic receptor antagonist agents in lieu of propranolol HCl and/or other antiemetic muscarinic receptor antagonist agents in lieu of scopolamine HBr.

### Example 2. Viscous Liquids & Semi-Solid Pharmaceutical Compositions

| Ingredients: | # 7 | | # 8 | | # 9 | | #10 | |
|---|---|---|---|---|---|---|---|---|
| Propranolol HCl | 600 | mg | 600 | mg | 600 | mg | 600 | mg |
| Scopolamine HBr, | 15 | mg | 15 | mg | 15 | mg | 15 | mg |
| Trihydrate | | | | | | | | |
| Glycerol | — | | — | | QS 15 g | | QS 15 g | |
| Propylene Glycol | 3.0 | ml | 3.0 | ml | 3.0 | ml | 3.0 | ml |

| | | | | | | | |
|---|---|---|---|---|---|---|---|
| Polyethylene Glycol | — | 4.5 | ml | 4.5 | ml | 4.5 | ml |
| (300) | | | | | | | |

| | | | | | | |
|---|---|---|---|---|---|---|
| Methyl Cellulose | — | 300 | mg | — | 300 | mg |
| (4000 Mpas) | | | | | | |

| Spira-Wash Gel | QS 15 g | — | — | — | |
|---|---|---|---|---|---|
| Sodium Bicarbonate | — | — | — | 30 | mg |

| | | | | |
|---|---|---|---|---|
| Purified Water | — | QS 15 g | — | — |

As examples of viscous liquid and semi-solid (e.g, gel, cream, or emulsion) pharmaceutical compositions for use in mucosal delivery (e.g., for rectal or vaginal application), formulations are prepared consisting of 40 mg/m propranolol HC and 1 mg/ml scopolamine HBr as the APIs. The inactive excipients are selected from; (a) alcoholic penetration enhancers and preservatives—propylene glycol and glycerol; (b) viscosity agents—polyethylene glycol, methyl cellulose, and Spira-Wash Gel™ base (PCCA); and (c) a buffering agent—sodium bicarbonate, for use in the rectum or in the vagina in post-menopausal women. Note that Spira-Wash Gel™ is an extemporaneous compounding base that contains polyethylene glycols and propylene glycol, among other ingredients. The dosage is 0.5 ml in an adult, with a delivered dose of 20 mg propranolol HCl and 0.5 mg scopolamine HBr.

The pH of the semi-solid formulations is ˜4.5 without buffering (e.g., sample #8) and ˜5.0-5.5 with bicarbonate buffering at a ratio of 1:20 of sodium bicarbonate to propranolol HCl (e.g., sample #10). The vagina of reproductive-age women is acidic (pH 3.8-4.4), and would not require a buffering agent, A buffering agent or agents (e.g, bicarbonate or phosphate) may be included to increase and buffer the pH toward the neutral pH of the rectum or post-menopausal vagina. A preservative agent may be included.

The viscosities of samples #9 and #10 were determined using a Brookfield viscometer at ca. 200-250 cP. The viscosities of samples #8-10 could be further increased by additional methylcellulose and/or higher molecular weight polyethylene glycol(s). Of the four samples, the most viscous is sample #7, containing Spira-Wash Gel (PCA) as the majority component.

The aqueous semi-solids (or “viscous liquids”) may be dispensed by a disposable single-dose applicator (e.g., delivers 0.5 or 1.0 ml for each pump action), such as a syringe-like device. Alternatively, semi-solid suppositories with higher viscosity may be prepared for manual insertion into the rectum or vagina.

In other viscous liquid or semi-solid compositions, one may substitute other beta adrenergic receptor antagonist agents in lieu of propranolol HCl and/or other antiemetic muscarinic receptor antagonist agents in lieu of scopolamine HBr.

### Example 3. Solid Pharmaceutical Compositions

| Ingredients: | # 11 | | #13 | | #14 | | #23 | | #25 | |
|---|---|---|---|---|---|---|---|---|---|---|
| Propranolol | 2.0 | g | 2.0 | g | — | | — | | — | |
| HCl | | | | | | | | | | |
| Atenolol | — | | — | | 2.5 | g | 1.83 | g | 3.05 | g |
| Scopolamine | 50 | mg | 25 | mg | 25 | mg | 18 | mg | 30 | mg |
| HBr, | | | | | | | | | | |
| Trihydrate | | | | | | | | | | |
| Mannitol | 3.5 | g | 3.34 | g | 2.99 | g | 2.86 | g | 0.85 | g |
| Starch | 1.4 | g | 1.4 | g | 1.4 | g | 1.2 | g | 1.0 | g |
| Sodium | 50 | mg | 200 | mg | 50 | mg | 60 | mg | 50 | mg |
| Bicarbonate | | | | | | | | | | |
| Magnesium | — | | 35 | mg | 35 | mg | 30 | mg | 25 | mg |
| Stearate | | | | | | | | | | |
| Total | 7.0 | g | 7.0 | g | 7.0 | g | 6.0 | g | 5.0 | g |

With regard to solid pharmaceutical compositions, powders, tablets, and capsules are prepared consisting of 10-80 mg propranolol HCl or 25-100 mg atenolol and 0.25-1.0 mg scopolamine HBr per dose. The inactive ingredients are selected from starch as the binding agent, mannitol as a sweetener, diluent, and dispersant, and sodium bicarbonate as a buffer and dispersant. Magnesium stearate may help in flow of the powder, especially in tablet-making using a mechanical tablet press. A preservative agent may be included in the formulation.

Tablets are formed by compression of the powder using a mechanical tablet press. For instance tablets without coatings are prepared with composition #11 at about 70 mg each, for use as sublingual or oral dose forms. At this size of tablet an adult dose would deliver 20 mg propranolol HC and 0.5 mg scopolamine HBr. Alternatively, tablets may be produced using a triturate tablet mold (e.g., from the vendor PCCA), wherein the triturated powder is moistened with 95% ethanol and then the wells are filled with the moistened composition, extruded from the wells, and dried. The intra-oral mucosal (e.g., sublingual) tablets dissolve rapidly under the tongue, for instance in less than one minute (e.g., solid dose forms #13 and 14). Propranolol HCl manifested an “acidic” and “medicine” taste as well as delayed paresthesia of the tongue in humans, whereas atenolol lacked these undesirable characteristics (i.e., comparison of solid dose forms #13 vs. 14). Sublingual (or buccal) tablets may be preferred for rapid disintegration, dissolution, absorption and increased bioavailability via the mucosal route. A sublingual tablet of #23 weighing ca. 82 mg delivers a dose of ca. 25 mg atenolol and ca. 0.25 mg of scopolamine HBr. Tablets may also be ingested orally, although it is rational to presume that absorption will be somewhat delayed and bioavailability will be somewhat reduced with many APIs, relative to the mucosal route.

As an alternative to compressed tablets, lyophilized tablets may also be prepared, with the advantage of lower density and increased rates of dispersion. Another alternative to compressed tablets, gelatin capsules (or other biocompatible and biodegradable capsules) may be filled with the powder. Gelatin capsules may be selected to disintegrate or dissolve rapidly, for instance within 15 minutes or more preferably within 5 minutes in the stomach. Gelatin capsules would likely delay, albeit slightly, the absorption and pharmacologic effects, relative to a comparable uncoated tablet or liquid dose form. When “time is of the essence” for symptomatic treatment a gelatin capsule would not be preferred.

The preferred routes of delivery for solid compositions are oral and sublingual, although in certain circumstances tablets may be used in other mucosal routes of delivery. In an embodiment, taste-masking flavors and sweeteners other than mannitol, or in addition to mannitol, may be included in the formulation, Powders may be dispensed in pre-dosed quantities to be mixed into a drink (e.g, water) for ingestion, Powders intended for ingestion might not require starch or another binding agent. Tablets may be ingested orally or applied to the oral mucosal for sublingual (or buccal) delivery. Capsules (e.g., gel capsules) filled with the pharmaceutical composition may be ingested orally or opened to pour the powder into a drink.

A buffering agent or agents (e.g., bicarbonate or phosphate) may be included in formulations to increase and buffer the pH to that of the oral cavity (˜6.2-7.4) for sublingual and/or buccal delivery. A buffering agent or agents might be included for oral medications (e.g, tablets or powders), although it should be noted that the stomach environment is acidic. Alkaline compositions could benefit from the use of an organic acid (e.g., citric acid) or buffer.

In other solid compositions, one may substitute other beta adrenergic receptor antagonist agents in lieu of propranolol HI or atenolol and/or other antiemetic muscarinic receptor antagonist agents in lieu of scopolamine HBr.

Examples of a preferred embodiment is a solid dosage form combination of 25 mg atenolol—0.2 mg scopolamine HBr for mucosal and/or oral administration using a variety of excipients, Compounded pharmaceutical compositions at these dosages have been been reported as effective and fast-acting p.r.n. anxiolytic treatments, when administered to patients in need of anti-anxiety treatments [15]-[21]. It is also reasonable to expect that higher and lower doses of atenolol and/or scopolamine in this combination may also be effective as anxiolytic compositions.

### Example 4. Method of Treating

Due to their high degree of pharmacologic activity and their low toxicity, together presenting a most favorable therapeutic index, the active principles of the present invention are administered to a subject or patient who is undergoing treatment for alcohol and/or drug addiction and is abstinent to the use of alcohol and/or drugs and is in need of treatment, alleviation, amelioration, palliation, or elimination of a symptom or symptoms of anxiety, wherein administration of the pharmaceutical compositions of the invention reduces a symptom or symptoms of anxiety, thereby reducing the risk of relapse to the use of alcohol and/or drugs in the patient.

The pharmaceutical compositions of the invention are administered concurrently, simultaneously, or in conjunction with a standard-of-care medication for the treatment of alcohol and/or drug addiction. Alternatively, the pharmaceutical compositions of the invention are administered without concurrently, simultaneously, or in conjunction with a standard-of-care medication for the treatment of alcohol and/or drug addiction.

The pharmaceutical compositions of the invention consist essentially of a therapeutically effective amount of at least one beta adrenergic receptor antagonist agent and/or pharmaceutically acceptable salts thereof, and at least one antiemetic muscarinic receptor antagonist agent and/or pharmaceutically acceptable salts thereof, and may comprise one or more pharmaceutically-acceptable excipients, carriers, or diluents, and is in a form for administration by an oral, sublingual, buccal, rectal, vaginal, transdermal, or parenteral route. Suitable dosage ranges are 0.1-400 milligrams, preferably 0.1-200 milligrams, and especially 0.1-100 milligrams, depending as usual upon the historic dosages in humans for the individual APIs and the exact mode of administration, form in which administered, the indication toward which the administration is directed, the subject involved and the body weight or surface area of the subject involved, and the preference and experience of the physician in charge. The active agents of the present invention are administered orally, mucosally (e.g., buccally, by nasal inhalation, or rectally), topically, or parenterally in dosage unit formulations containing conventional non-toxic pharmaceutically acceptable carriers. It may be desirable to use the mucosal route to hasten the pharmacologic effects. Furthermore, it may be desirable to use the oral route.

A specific dose naturally varies depending on the dosage procedure, the conditions of a patient or a subject such as age, body weight, sex, sensitivity, feed, dosage period, drugs used in combination, seriousness of the symptoms. The appropriate dose and dosage times under certain conditions can be determined by the test based on the above-described indices but may be refined and ultimately decided according to the judgment of the practitioner and each patient's circumstances (age, general condition, severity of symptoms, sex, etc.) according to standard clinical techniques.

Toxicity and therapeutic efficacy of the pharmaceutical compositions of the present invention consisting essentially of a therapeutically effective amount of at least one beta adrenergic receptor antagonist agent and/or pharmaceutically acceptable salts thereof, and at least one antiemetic muscarinic receptor antagonist agent and/or pharmaceutically acceptable salts thereof, can be determined by standard pharmaceutical procedures in experimental animals, e.g., by determining the LD₅₀ (the dose lethal to 50% of the population) and the ED₅₀ (the dose therapeutically effective in 50% of the population). The dose ratio between therapeutic and toxic effects is the therapeutic index and it can be expressed as the ratio ED₅₀/LD₅₀.

### Example 5. Treatment of Patients Undergoing Therapy for Alcohol and/or Drug Addiction

The present invention provides a self-administered p.r.n. symptomatic therapy taken by a patient undergoing treatment for alcohol and/or drug addiction at the time of an episode of panic attack (PA) or anxiety, or in anticipation of a panic or anxiety episode, or before or at the time of a “trigger” circumstance for PA or anxiety, or a alcohol-associated cue, or a drug-associated cue. The pharmaceutical compositions of the invention may be taken by a patient undergoing treatment for alcohol and/or drug addiction on a p.r.n. basis to reduce the risk of an episode of PA or anxiety. The pharmaceutical composition having an effective amount of a combination of an antiemetic muscarinic receptor antagonist drug and a beta adrenergic receptor antagonist drug is administered and temporarily alleviates some of the symptoms of PA and anxiety at the time of a symptomatic episode or in anticipation of an episode.

Compositions of the present invention may also be of value as a supportive p.r.n. therapy during cognitive behavioral therapy (CBT) or other forms of counseling for the treatment of alcohol and/or drug addiction. Benjamin et al., 2017 is a case report wherein compounded atenolol—scopolamine HBr was prescribed by a psychiatrist and used effectively as a p.r.n. anxiolytic in conjunction with psychotherapy in a patient with a history of substance abuse [15].

The compositions may provide anxiolytic benefit without cognitive impairment while learning or reinforcing desirable behaviors. The dual drug therapies may also be used p.r.n. when the patients experience acute anxiety episodes between sessions of CBT or other forms of counseling.

The patients undergoing therapy for alcohol and/or drug addiction and are abstinent to alcohol and/or drug use, at the time of an episode of panic attack (PA) or anxiety or in anticipation of an episode of PA or anxiety, may self-medicate by mucosal (e.g., sublingual, buccal, rectal, vaginal, or nasal) or oral routes, using the pharmaceutical compositions of the invention. Mucosal routes of drug delivery provide more rapid relief of symptoms compared to orally-ingested pharmaceutical compositions. Mucosal routes abrogate first-pass metabolism by the liver. Therefore, sublingual/buccal (or other mucosal) routes of administration provide immediate or more immediate relief when a patient has an episode of PA or anxiety, as well as in abrogating an episode of PA or anxiety upon encountering a trigger circumstance or drug-associated cue in advance of the expected symptoms.

The patients are treated p.r.n. mucosally (e.g., sublingually) or orally with solid dose compositions (e.g., tablets) consisting essentially of scopolamine at 0.1-0.5 mg in combination with atenolol at 25-50 mg or propranolol at 20-40 mg, as representative examples, Other combinations of APs and doses are also envisioned. Patients will experience beneficial abrogation or reduction in somatic and/or psychic (CNS) symptoms of PA and/or anxiety. The treatment provides an anxiolytic and/or calming effect. The beneficial effects (and side effects, if any) are hastened by the sublingual route of administration relative to an oral route of administration. Asa specific example, a healthy adult subject who is currently receiving treatment for a substance use disorder, is abstinent from alcohol and/drug use, and is experiencing symptoms of anxiety and thoughts of relapsing to drug use, is administered a pharmaceutical composition consisting essentially of a therapeutically effective amount of at least one beta adrenergic receptor antagonist agent and/or pharmaceutically acceptable salts thereof, and at least one antiemetic muscarinic receptor antagonist agent and/or pharmaceutically acceptable salts thereof. A calming effect commenced in less than 60 minutes (and preferably less than 30 or 15 minutes) and lasted for multiple hours in duration, and without sedation. Dryness of the mouth was a side effect.

A pharmaceutical composition consisting essentially of a therapeutically effective amount of at least one beta adrenergic receptor antagonist agent and/or pharmaceutically acceptable salts thereof, and at least one antiemetic muscarinic receptor antagonist agent and/or pharmaceutically acceptable salts thereof, is superior to treatments by the alternatives—most notably benzodiazepines (as well as opioids, opiates, or cannabinoids), particularly in subjects who are prone to, exhibit alcohol and/or drug dependency, and/or are being treated for drug and/or alcohol addiction and are abstinent to alcohol and/or drug use, especially given that the APIs of the present invention are non-dependent, non-addicting, and not Controlled Substances that are often abused.

Patients who are undergoing treatment for a substance use disorder are treated with a pharmaceutical composition consisting essentially of a therapeutically effective amount of at least one beta adrenergic receptor antagonist agent and/or pharmaceutically acceptable salts thereof, and at least one antiemetic muscarinic receptor antagonist agent and/or pharmaceutically acceptable salts thereof, in conjunction with a standard-of-care medicine for alcohol addiction and/or drug addiction, including opioid addiction, which can manifest the common sympathetic and parasympathetic symptoms of panic and anxiety. Upon administration, the pharmaceutical compositions consisting essentially of a therapeutically effective amount of at least one beta adrenergic receptor antagonist agent and/or pharmaceutically acceptable salts thereof, and at least one antiemetic muscarinic receptor antagonist agent and/or pharmaceutically acceptable salts thereof, affect the symptoms of panic and/or anxiety, and thereby reduces the risk for relapse in the patient.

The present invention produces a calming effect in individuals, as discerned by the treated individual and/or observed by an objective observer. As examples the self-perceived calming effect has been reported for patients when treated either mucosally or orally by a combination of a beta blocker and an antimuscarinic agent [15]-[21] Furthermore, some of the reported patients treated in this manner for anxiety symptoms had clinical histories of SUDs, such as alcohol, opioids, and/or benzodiazepines [15]-[21]. Therefore, a fixed dose combination of atenolol—scopolamine HBr has been shown to be safe and effective in treating anxiety symptoms in SUD patients.

The subjective calming effect of atenolol—scopolamine HBr has been confirmed in patients using anxiety assessment instruments, for instance the Beck Anxiety Instrument-State (BAI-S) and symptom questionnaires [15]-[21]. In addition to subjective endpoints of anxiolytic effectiveness, beneficial objective endpoints have also been assessed in patients, such as heart rate, systolic blood pressure, and diastolic blood pressure [15]-[21]. These results from physician-sponsored studies provide evidence of the drug combination acting upon both parasympathetic and sympathetic symptoms of acute anxiety.

The pharmaceutical composition consisting essentially of a therapeutically effective amount of at least one beta adrenergic receptor antagonist agent and/or pharmaceutically acceptable salts thereof, and at least one antiemetic muscarinic receptor antagonist agent and/or pharmaceutically acceptable salts thereof, for instance scopolamine plus a beta blocker (e.g., atenolol or propranolol), affects most or all of the symptoms typically associated with panic attack (PA) and/or anxiety that are coincident and/or manifest during breakthrough anxiety during standard-of-care treatments for drug addiction and alcohol addiction. Furthermore, an anxiolytic or calming effect diminishes the number and magnitude of the symptoms of anxiety or breakthrough anxiety experienced in patients who are undergoing treatment using current standard-of-care therapies, or have been detoxified under current standard-of-care therapies, and reduces the risk of relapse and/or prevents relapse in those patients.

### Example 6. Clinical Trials

Clinical trials are designed to study the combination therapies of the present invention. One type of trial design is termed as “in life” usage. The patients, who are preferably or are undergoing treatment for a substance use disorder and are compliant with a deterrent standard-of-care medicine, at the time of an episode of panic attack (PA) or anxiety, or in anticipation of an episode, or before or at the time of a “trigger” circumstance or drug-associated cue, self-medicate by administering a pharmaceutical composition consisting essentially of an antiemetic muscarinic receptor antagonist drug and a beta adrenergic receptor antagonist drug and record by written or electronic means their specific symptoms of episodes, number and severity of symptoms of episodes, timing and frequency of use of the therapy (either at onset or in anticipation of), perceived benefits, for example, suppression of the patient's cravings or desire for alcohol and/or drugs, perceived side-effects, etc. The patient's self-assessment(s) at specified time points can include analog (e.g., visual analog scale) or digital (e.g., binary or discrete unit scale) assessment tools. Objective measurements, such as heart rate, systolic blood pressure, and diastolic blood pressure may also be obtained in the clinic or “in life” using appropriate instruments.

This “in life” trial design mimics how the combination therapy of a standard-or-care medicine and a pharmaceutical composition consisting essentially of a therapeutically effective amount of at least one beta adrenergic receptor antagonist agent and/or pharmaceutically acceptable salts thereof, and at least one antiemetic muscarinic receptor antagonist agent and/or pharmaceutically acceptable salts thereof, is used in normal settings in the patient's life. For example, the patient may be receiving a standard-of-care medicine for alcohol and/or drug addiction. The trials are “open label” in design, where the identity of the combination is disclosed to the physicians and patients. Examples of “open label” studies of the present invention have been performed in psychiatry and pain management settings [15]-[21].

Alternatively, a placebo of similar physical properties, yet lacking the beta adrenergic receptor antagonist agent and antiemetic muscarinic receptor antagonist agent, is used as a control in “blinded” studies. The patients can be studied in a parallel trial design. The patients can also be studied in a cross-over trial design, in which the first period of time is on either the therapy or the placebo. Then, during the next period of time the patient is switched over to the alternative. The results are collected at later time points and analyzed by statistical methods to demonstrate efficacy and/or safety of the pharmaceutical compositions of the invention.

Clinical trials are designed for patients affected by alcohol addiction and/or withdrawal, drug addiction and/or withdrawal. The studies are conducted either “in life” (e.g, “open label” use of the compositions) or in a clinic setting. When desired, the trials may be placebo controlled and blinded to the investigators and/or patients. The dosing is p.r.n., and intended for occasional use in the context of the treatment for alcohol and/or drug addiction to prevent relapse to alcohol and/or drug use. In an embodiment, the dosing is p.r.n., and intended for daily use as needed by the patient in the context of the treatment for alcohol and/or drug addiction to prevent relapse to alcohol and/or drug use.

A clinical endpoint can be the reduction in number of patients that relapse over days, weeks, or months when treated with the beta blocker-antimuscarinic combination of the present invention versus placebo. The clinical trial may be designed either without or with coincident standard-of-care treatments for SUDs, although the latter is preferable.

Another clinical endpoint can be the increase in number of days, weeks, or months before the patients relapse when treated with the beta blocker-antimuscarinic combination of the present invention versus placebo. The clinical trial may be designed either without or with coincident standard-of-care treatments for SUDs, although the latter is preferable.

Another clinical endpoint can be the patients' self-assessments of their likelihood to remain abstinent (or to relapse) from alcohol and/or drugs when treated with the beta blocker—antimuscarinic combination of the present invention, in view of anticipating having available at their convenience an effective anti-anxiety medication that can be administered p.r.n. (as needed) during their recovery or maintenance from SUDs.

As a specific example, a parallel group clinical trial has been designed to compare a fixed dose combination drug to placebo in reducing the rate of OUD relapse at selected timepoints (e.g. weeks and months), and wherein the opioid abstinent patients have been detoxified and are compliant with a medical assisted treatment program (MAT), for instance buprenorphine, buprenorphine—naloxone, or naltrexone. Reference rates of relapse while compliant to MAT are known and instruct statistical power analyses for demonstration of clinical effectiveness by the anxiolytic combination drug treatment.

A patient undergoing treatment for alcohol and/or drug addiction self-administers either mucosally (e.g., sublingually) or orally a composition consisting essentially of scopolamine HBr (e.g, at preferably ca. 0.1-0.5 mg) in conjunction with a beta adrenergic receptor antagonist (e.g, atenolol at preferably ca. 25-50 mg or propranolol HCl at preferably ca. 20-40 mg). A preferred embodiment is a combination of scopolamine HBr at ca. 0.2 mg with atenolol at ca. 25 mg. Within an hour or less the patient and/or observer perceives benefit(s) with regard to anxiety symptoms. The therapeutic benefit(s) persist for multiple hours, for example, as long as hours, 8 hours, or even 24 hours. Following treatment the patient experiences an anxiolytic or calming effect that results in the perception of relief from anxiety, in addition to a reduction in cardiovascular symptoms (e.g, elevated heart rate, palpitations, and elevated blood pressure) due to epinephrine in the circulation. These benefits can be achieved at doses of the APIs that are non-sedating. The anxiolytic or calming effect can improve voluntary and involuntary motor control, task performance, cognition, memory, avoidance of harm or danger, and/or reduce fear, and thereby reduce the risk of relapse to alcohol and/or drugs.

The pharmaceutical compositions consisting essentially of a therapeutically effective amount of at least one beta adrenergic receptor antagonist agent and/or pharmaceutically acceptable salts thereof, and at least one antiemetic muscarinic receptor antagonist agent and/or pharmaceutically acceptable salts thereof, are administered by subjects who were deemed by the licensed medical professionals to be addicted to alcohol and/or drugs and are in need of p.r.n. anxiolytic treatments. The pharmaceutical compositions of the invention are advantageously administered by the oral and/or mucosal (e.g. sublingual) routes of administration for rapid-acting anxiolytic benefit in the patient.

### Example 7. Patient Treatment

A patient having been diagnosed with and/or being treated for a substance use disorder and is affected by symptoms of anxiety and/or panic, is beginning to experience symptoms of a panic attack or acute anxiety (e.g., elevated heart rate, palpitations, or nausea), or is anticipating symptoms of panic and/or anxiety and self-administers p.r.n. a pharmaceutical composition consisting essentially of propranolol and scopolamine or atenolol and scopolamine. Some or all of the symptoms associated with panic and/or anxiety are suppressed and/or alleviated in the patient. The medication produces an anxiolytic and/or calming effect in the patient. Cravings for alcohol and/or drugs by the patient is suppressed and/or temptations to alcohol and/or drug use is removed from the patient.

### Example 8. Patient Treatment

A patient diagnosed with and or being treated for a substance use disorder and is affected by the symptoms of panic and/or anxiety, is exposed to a trigger circumstance that has formerly resulted in a panic or anxiety or a drug-cue and immediately self-administers p.r.n. a pharmaceutical composition consisting essentially of propranolol and scopolamine or atenolol and scopolamine. As a result of treatment, some or all of the symptoms associated with panic or anxiety are prevented and/or alleviated in the patient. The medication produces an anxiolytic and/or calming effect and cravings or desire for alcohol and/or drugs by the patient is suppressed and/or temptations to alcohol and/or drug use is removed from the patient.

### Example 9. Patient Treatment

In certain panic- and acute anxiety-prone patients the pharmaceutical compositions of the invention consisting essentially of an antiemetic muscarinic receptor antagonist drug and a beta adrenergic receptor antagonist drug are sublingually or orally administered in advance of an anticipated trigger circumstance or drug-associated cue which elicits symptoms of a panic and/or anxiety. When the patient anticipates a trigger or symptoms in 15-60 minutes in the future, then self-medication is started 15-60 minutes prior to the expected trigger or symptoms. Furthermore, when “time is of the essence” the mucosal route(s) of delivery are preferred (e.g., sublingual). For instance, if a patient desires the effect of the medication in 30 minutes or less, the mucosal route(s) of delivery are preferred, whereas if the patient desires the effect beyond 30 minutes then the oral route of delivery may be preferred. It follows that an oral gelatin capsule would delay somewhat the therapeutic benefits, and is not preferred for rapid therapy.

The efficacious benefit of a single administered dose will last for hours, provided sufficient blood levels of the antiemetic muscarinic receptor antagonist and/or the beta adrenergic receptor antagonist APIs are sustained. The overt effects of a single dose may diminish over the course of a day, as the APIs are progressively metabolized and/or excreted. Note that both scopolamine and propranolol are metabolized by the liver, whereas atenolol is not. Thus, an impairment of hepatic functions might prolong the effects of the certain drug combinations. The blood levels of the APIs are influenced by multiple pharmacologic and physiologic parameters, such as absorption, distribution, metabolism, and excretion (ADME), as well as other individual patient genetic and environmental conditions. It follows that the blood levels of the APIs are expected to vary somewhat from patient to patient. This variability could affect efficacy.

### Example 10, Patient Treatment

Patients affected by alcohol addiction and/or withdrawal, drug addiction and/or withdrawal, and optionally being treated with a standard-of-care medicine for addiction are treated in a similar manner using the pharmaceutical compositions of the present invention. Following treatment the patient experiences an anxiolytic and/or calming effect that results in the perception of relief from anxiety, in addition to a reduction in cardiovascular symptoms (e.g., elevated heart rate, palpitations, and elevated blood pressure) due to epinephrine in the circulation. The anxiolytic or calming effect may improve cognition, memory, voluntary and involuntary motor control, task performance, avoidance of harm or danger, and/or may reduce fear, reduce drug cravings and reduce risk of relapse to drug and/or alcohol use. The pharmaceutical compositions of the invention when administered coincidentally with a standard-of-care medicine reduce the number and/or severity of the symptoms associated with symptoms of panic and/or anxiety in such patients and augment the standard-of-care therapy such that the patients feel better and will desire to continue with therapy. The benefit of the at least one beta adrenergic receptor antagonist agent and/or pharmaceutically acceptable salts thereof, and at least one antiemetic muscarinic receptor antagonist agent and/or pharmaceutically acceptable salts thereof, is superior to an antiemetic muscarinic therapy alone or a beta blocker therapy alone. The pharmaceutical compositions consisting essentially of a therapeutically effective amount of at least one beta adrenergic receptor antagonist agent and/or pharmaceutically acceptable salts thereof, and at least one antiemetic muscarinic receptor antagonist agent and/or pharmaceutically acceptable salts thereof, are superior to treatments by the alternatives—benzodiazepines, opioids, opiates, or cannabinoids, especially given that the APIs of the present invention are non-addicting and not Controlled Substances that are often abused.

### Example 11. Patient Treatment

Subjects diagnosed with Opioid Use Disorder who are already detoxified and compliant with an opioid deterrent standard-of-care medicine and are exhibiting symptoms of anxiety, self-administer p.r.n a pharmaceutical composition consisting essentially of a combination of at least one beta adrenergic receptor antagonist agent and/or pharmaceutically acceptable salts thereof, and at least one antiemetic muscarinic receptor antagonist agent and/or pharmaceutically acceptable salts thereof. The pharmaceutical composition consists essentially of propranolol and scopolamine or atenolol and scopolamine. Some or all of the symptoms associated with anxiety are suppressed and/or alleviated in the patient. The medication produces an anxiolytic and/or calming effect of the subject. Cravings or desire for drugs by the patient is suppressed and/or temptations to drug use is removed from the subject.

### Example 12. Patient Treatment

Subjects having an opioid addiction are treated with an opioid deterrent standard-of-care medicine and are exhibiting symptoms of anxiety with thoughts of returning to opioid use, self-administer p.r.n a pharmaceutical composition consisting essentially of a combination of at least one beta adrenergic receptor antagonist agent and/or pharmaceutically acceptable salts thereof, and at least one antiemetic muscarinic receptor antagonist agent and/or pharmaceutically acceptable salts thereof. The pharmaceutical composition consists essentially of propranolol and scopolamine or atenolol and scopolamine.

Following treatment the patient experiences an anxiolytic and/or calming effect that results in the perception of relief from anxiety, in addition to a reduction in cardiovascular symptoms (e.g., elevated heart rate, palpitations, and elevated blood pressure) due to epinephrine in the circulation. The anxiolytic or calming effect may improve cognition, memory, voluntary and involuntary motor control, task performance, avoidance of harm or danger, and/or reduces cravings or desire for alcohol and/or drugs by the patient.

### Example 13. Patient Treatment

The literature reference Benjamin & Dooley (2019) [18] demonstrates treatment of eight patients who were diagnosed by a psychiatrist with various anxiety disorders and, in an open label study, were administered atenolol 25 mg-scopolamine HBr 0.2 mg. Three of the patients were being treated in a residential setting for SUDs, such as alcohol or opioids. Six of the eight were responders, including one diagnosed with Opioid and Benzodiazepine Use Disorder. Three of the six responders noted that the combination drug compared favorably to benzodiazepines based upon their prior experiences. Thus, they perceived a benzodiazepine-like calming effect.

Furthermore, the literature reference Dooley, et al. (2019) [19] summarizes the results of physician-sponsored studies to date of compounded PanX® atenolol—scopolamine HBr in 22 patients, which demonstrated a fast-acting and self-perceived calming effect orally within <60 minutes or mucosally (orally-disintegrating) within <30 minutes. The self-perceived calming effect was reported by 19 of 22 patients. It persisted for multiple hours, with some patients noting that the self-awareness of a calming effect lasted up to 8 hours. Minor and well tolerated side effects were noted (e.g., dry mouth, mild sedation), and occurred in about half of the patients. At least six of the 22 patients commented that atenolol 2 mg-scopolamine HBr 0.2 mg produced anxiolytic effects similar to that of benzodiazepines from their prior experiences.

### Example 14. Patient Treatment

Subjects affected by SUDs (either alcohol or drugs) and one or more symptoms of anxiety that may predispose the subject to relapse, are diagnosed by a medical practitioner (e.g, MD, DO, CNRP, PA) and are treated with a pharmaceutical composition consisting essentially of a combination of at least one beta adrenergic receptor antagonist and/or pharmaceutically acceptable salts thereof, and at least one antiemetic muscarinic receptor antagonist agent and/or pharmaceutically acceptable salts thereof. The pharmaceutical composition produces a rapid self-perceived calming (anxiolytic) effect in less than 60 minutes and persists for hours in the SUD subject.

The reduction in anxiety symptom severity and/or number of symptoms provides a beneficial advantage that helps reduce the subject's risk of relapse to alcohol and/or drugs, thus increasing longevity in subjects who are prone to overdoses or the cumulative chronic health problems due to alcohol or drugs. The subject affected by an SUD may self-report of an anxiolytic benefit by this treatment with the self-perception of his/her expectation of increased likelihood of coping in maintaining abstinence (relapse-free condition). Alternatively a medical practitioner may report the same on behalf of the subject affected by an SUD. The subject's self-reporting of an anxiolytic benefit may mirror that of prior experience with a benzodiazepine anxiolytic, Given that anxiety is a key factor contributing to the incidence of SUP relapse, reductions in anxiety symptom severity and/or number in SUD subjects reduce drug or alcohol craving and/or drug- or alcohol-seeking behavior, thereby reducing the probability of relapse, and thus ultimately resulting in an increase in abstinence duration and/or longevity. The reduction in anxiety symptom severity and/or number without the use of a benzodiazepine anxiolytic in SUD subjects is advantageous.

The present invention is not to be limited in scope by the specific embodiments described herein. Indeed, various modifications of the invention in addition to those described herein will become apparent to those skilled in the art from the foregoing description.

All patents, applications, publications, test methods, literature, and other materials cited herein are hereby incorporated by reference. The disclosures of U.S. Pat. Nos. 9,446,030; 9,517,231; and 9,616,052, are hereby incorporated by reference in their entirety.

### REFERENCES

1, Brandon, T. H., J. I. Vidrine, and E. B. Litvin, Relapse and relapse prevention. Annu Rev Clin Psychol, 2007, 3: p. 257-84.
2. Sinha, R., New findings on biological factors predicting addiction relapse vulnerability. Curr Psychiatry Rep, 2011. 13(5): p, 398-405.
3. Sinha, R., Chronic stress, drug use, and vulnerability to addiction. Ann N Y Acad Sci, 2008, 1141: p. 105-30.
4. Milivojevic, V, and R. Sinha, Central and Peripheral Biomarkers of Stress Response for Addiction Risk and Relapse Vulnerability, Trends Mol Med, 2018.24(2) p. 173-186.
5. Willinger, U., et al., Anxiety as a predictor of relapse in detoxified alcohol-dependent patients. Alcohol, 2002. 37(6): p, 609-12.
6. Sinha, R, et al, Effects of adrenal sensitivity, stress- and cue-induced craving, and anxiety on subsequent alcohol relapse and treatment outcomes. Arch Gen Psychiatry, 2011. 68(9): p. 942-52.
7. Oliva, F, et al, Gender Differences in Anxiety and Depression before and after Alcohol Detoxification: Anxiety and Depression as Gender-Related Predictors of Relapse. Eur Addict Res, 2018. 24(4): p, 163-172
8. Sinha, R., How does stress lead to risk of alcohol relapse? Alcohol Res, 2012. 34(4): p. 432-40,
9. Smith, J. P. and C. L. Randall, Anxiety and alcohol use disorders: comorbidity and treatment considerations. Alcohol Res, 2012, 34(4): p, 414-31.
10. Hyman, S. M., et al., Stress and drug-cue-induced craving in opioid-dependent individuals in naltrexone treatment, Exp Clin Psychopharmacol, 2007. 15(2): p. 134-43.
11. Hermes, G., et al., Lofexidine in Combination with Oral Naltrexone for Opioid Use Disorder Relapse Prevention: A pilot Randomized, Double-Blind, Placebo-Controlled Study. Am J Addict, 2019.
12. Bonn-Miller, M. O. and R. H. Moos, Marijuana discontinuation, anxiety symptoms, and relapse to marijuana, Addict Behav, 2009, 34(9): p. 782-5,
13. Zvolensky, M. J, et al., Anxiety sensitivity and anxiety and depressive symptoms in the prediction of early smoking lapse and relapse during smoking cessation treatment. Nicotine Tob Res, 2009.11(3): p, 323-31.
14. Zvolensky, M. J, et al, Anxiety sensitivity and early relapse to smoking: a test among Mexican daily, low-level smokers. Nicotine Tob Res, 2007, 9(4): p. 483-91.
15. Benjamin, A., Mollner, A., & Dooley, T. P. (2017). Atenolol—Scopolamine Combination Drug Decreases Acute Anxiety Post Trauma Therapy Session. Annals of Depression and Anxiety, 4(2), 1086,
16. Benjamin, A. B, & Dooley, T. P. (2017a). Beta Blocker—Scopolamine Combination Drug Decreases Acute Anxiety in a PTSD Patient—A Case Report ARC Journal of Psychiatry, 2(3), 1-4,
17. Benjamin, A. B., & Dooley, T. P. (2017b). A compounded, nonbenzodiazepine option for treating acute anxiety. Current Psychiatry, 16(12), el-2.
18. Benjamin, A. B., & Dooley, T. P. (2019). Anxiolytic Benefits of Compounded Atenolol—Scopolamine in Eight Patients in Psychiatry. Personalized Medicine in Psychiatry. https://doi.org:10.1016/j.pmip.2019.10.001.
19. Dooley, T. P., Benjamin, A. B., and Thomas, T. (2019) “Treating Anxiety with a Beta Blocker—Antimuscarinic Combination: A Review of Compounded Atenolol—Scopolamine”. Clinical Psychiatry 5(3):63, dot:10.36848/2471-9854.5.1.63.
20. Thomas, T., Benjamin, A. B., & Dooley, T. P. (2017). Treatment of Acute Anxiety Episodes in Patients Using a Fast-Acting Beta Blocker—Scopolamine Combination Drug. Annals of Depression and Anxiety, 4(2), 1088.
21. Thomas, T., & Dooley, T. P. (2018). Treatment of Anxiety Prior to a Medical Procedure using an Atenolol—Scopolamine Combination Drug. Journal of Depression and Anxiety, 7(2), 303. doi:10.4172/2167-1044.1000303.

1. A method for reducing the occurrence of relapse to alcohol and/or drug use in a subject comprising, administering to a subject having a substance use disorder and who is abstinent to alcohol and/or drug use, a pharmaceutical composition consisting essentially of a combination of at least one beta adrenergic receptor antagonist agent and/or pharmaceutically acceptable salts thereof, and at least one antiemetic muscarinic receptor antagonist agent and/or pharmaceutically acceptable salts thereof, together with one or more pharmaceutically acceptable excipients, wherein the at least one beta adrenergic receptor antagonist agent and the at least one antiemetic muscarinic receptor antagonist agent are combined in amounts therapeutically effective for treatment of symptoms of anxiety, thereby reducing the risk of relapse to alcohol and/or drug use in the subject.

2. The method of claim 1, wherein the pharmaceutical composition is administered pro re nata (p.r.n.) by the subject.

3. The method of claim 1, wherein administration occurs when the subject anticipates symptoms of anxiety, or at the time of a trigger circumstance for anxiety, a drug-associated cue, an alcohol-associated cue, or during symptoms of anxiety.

4. The method of claim 1, wherein the subject having a substance use disorder is being detoxified from alcohol and/or drugs, or has been detoxified from alcohol and/or drugs.

5. The method of claim 1, wherein the subject is coincidentally receiving a standard-of-care medicine for alcohol addiction and/or drug addiction.

6. The method of claim 5, wherein the standard-of-care medicine is a deterrent to alcohol or drug use.

7. The method of claim 5, wherein the standard-of-care medicine is selected from the group consisting of an opioid antagonist, and opioid agonist, and a mixed opioid agonist/antagonist.

8. The method of claim 1, wherein the subject having a substance use disorder is addicted to one or more substances, and the substances consist of alcohol and drugs selected from opioids, opiates, and cocaine.

9. The method of claim 1, wherein the pharmaceutical composition is in a form for mucosal, sublingual buccal, rectal, vaginal, nasal or oral administration.

10. The method of claim 9, wherein the pharmaceutical composition is in a form selected from the group consisting of a spray, an elixir, a solution, a suspension, an emulsion, a get a cream, a gum, a powder, a tablet, a capsule, a troche, a suppository, a pill, and a film.

11. The method of claim 1, wherein the at least one beta adrenergic receptor antagonist agent is selected from the group consisting of propranolol, atenolol, pindolol, nadolol, nebivolol, and/or pharmaceutically acceptable salts thereof, and Eucommia extract or an active pharmaceutical ingredient of Eucommia, and wherein the at least one antiemetic muscarinic receptor antagonist agent is selected from the group consisting of scopolamine, diphenhydramine, promethazine, meclizine, hydroxyzine, and/or pharmaceutically acceptable salts thereof.

12. The method of claim 1, wherein the at least one beta adrenergic receptor antagonist agent is propranolol, atenolol, pindolol, nadolol, nebivolol, and/or pharmaceutically acceptable salts thereof, in an amount of about 10 to 100 mg/dose for an adult, and wherein the at least one antiemetic muscarinic receptor antagonist agent is scopolamine, and/or pharmaceutically acceptable salts thereof, in an amount of about 0.05 to 1 mg/dose for an adult.

13. A method of treating a subject with an opioid addiction comprising administering a combination of at least one standard-of-care medicine for opioid addiction and a pharmaceutical composition consisting essentially of a combination of at least one beta adrenergic receptor antagonist agent and/or pharmaceutically acceptable salts thereof, and at least one antiemetic muscarinic receptor antagonist agent and/or pharmaceutically acceptable salts thereof, together with one or more pharmaceutically acceptable excipients, wherein the at least one beta adrenergic receptor antagonist agent and the at least one antiemetic muscarinic receptor antagonist agent are combined in amounts therapeutically effective for treatment of symptoms of anxiety in a subject and wherein the pharmaceutical composition is in a form for mucosal sublingual buccal, rectal, vaginal, nasal, or oral p.r.n. administration by the subject anticipating and/or experiencing symptoms of anxiety incident to treatment and/or recovery from opioid addiction, opioid withdrawal, trigger circumstances or drug-associated cues.

14. The method of claim 13, wherein the at least one beta adrenergic receptor antagonist agent is selected from the group consisting of propranolol, atenolol, pindolol, nadolol nebivolol, and/or pharmaceutically acceptable salts thereof, and Eucommia extract or an active pharmaceutical ingredient of Eucommia, and wherein the at least one antiemetic muscarinic receptor antagonist agent is selected from the group consisting of scopolamine, diphenhydramine, promethazine, meclizine, hydroxyzine, and/or pharmaceutically acceptable salts thereof.

15. The method of claim 13, wherein the at least one beta adrenergic receptor antagonist agent is propranolol, atenolol, pindolol, nadolol, nebivolol, and/or pharmaceutically acceptable salts thereof, in an amount of about 10 to 100 mg/dose for an adult, and wherein the at least one antiemetic muscarinic receptor antagonist agent is scopolamine, and/or pharmaceutically acceptable salts thereof, in an amount of about 0.05 to 10 mg/dose for an adult, and wherein the doses are lower for an adolescent or child.

16. The method of claim 13, wherein the standard-of-care medicine is selected from the group consisting of an opioid antagonist, and opioid agonist, and a mixed opioid agonist/antagonist.

17. A combination for treating alcohol and/or drug addiction comprising a standard-of-care medicine for alcohol and/or drug addiction and a pharmaceutical composition consisting essentially of at least one beta adrenergic receptor antagonist agent and/or pharmaceutically acceptable salts thereof, and at least one antiemetic muscarinic receptor antagonist agent and/or pharmaceutically acceptable salts thereof, wherein the at least one beta adrenergic receptor antagonist agent and the at least one antiemetic muscarinic receptor antagonist agent are combined in amounts therapeutically effective for treatment of symptoms of anxiety in a subject and wherein the pharmaceutical composition is in a form for mucosal, sublingual; buccal, rectal, vaginal, nasal, or oral p.r.n. administration by the subject anticipating and/or experiencing symptoms of anxiety incident to treatment and/or recovery from alcohol and/or drug addiction, alcohol and/or drug withdrawal, trigger circumstances or drug-associated cues.

18. The combination of claim 17, wherein the standard-of-care medicine is selected from the group consisting of an opioid antagonist, an opioid agonist, a mixed opioid agonist/antagonist, acamprosate, or disulfiram.

19. The combination of claim 17, wherein the at least one beta adrenergic receptor antagonist agent is selected from the group consisting of propranolol, atenolol, alprenolol, acebutolol, betaxolol, bisoprolol bucindolol, celiprolol, nadolol, sotalol, esmolol, carteolol, carvedilol, mepindolol, nebivolol, oxprenolol, penbutolol pindolol, landiolol, metoprolol, timolol, labetolol, and/or pharmaceutically acceptable salts thereof, and Eucommia extract, an active pharmaceutical ingredient of Eucommia, and/or pharmaceutically acceptable salt thereof.

20. The combination of claim 17, wherein the at least one antiemetic muscarinic receptor antagonist agent is selected from the group consisting of scopolamine, diphenhydramine, meclizine, buclizine, cyclizine, hydroxyzine, pirenzepine, benztropine (benzatropine), butylscopolamine, methylscopolamine, doxylamine, promethazine, trihexyphenidyl, orphenadrine, and/or pharmaceutically acceptable salts thereof.

21. The combination of claim 17, wherein the at least one beta adrenergic receptor antagonist agent and/or pharmaceutically acceptable salts thereof, is in an amount of about 10 to 100 mg/dose, and wherein the at least one antiemetic muscarinic receptor antagonist agent and/or pharmaceutically acceptable salts thereof, is scopolamine and/or pharmaceutically acceptable salts thereof in an amount of about 0.05 to 10 mg/dose, and/or is not scopolamine and/or pharmaceutically acceptable salts thereof in an amount of about 10 to 100 mg/dose.

22. The combination of claim 17, wherein the at least one antiemetic muscarinic receptor antagonist agent is scopolamine and/or pharmaceutically acceptable salts thereof, and wherein the at least one beta adrenergic receptor antagonist agent and the at least one antiemetic muscarinic receptor antagonist agent are combined in amounts therapeutically effective for p.r.n. symptomatic treatment in a ratio in a range of 300:1 to 40:1 (beta adrenergic receptor antagonist agent:antiemetic muscarinic receptor antagonist agent).
